(19) United States
(12) Patent Application Publication
(10) Pub. No.: US 2026/0166041 A1
(43) Pub. Date: Jun. 18, 2026

(54) COMPOSITIONS AND METHODS FOR TREATING A SEIZURE DISORDER IN A SELECTED SUBJECT

(71) Applicants: The Children's Medical Center Corporation, Boston, MA (US); The General Hospital Corporation, Boston, MA (US); The Brigham and Women's Hospital, Inc., Boston, MA (US)

(72) Inventors: Christopher A. Walsh, Boston, MA (US); Kristopher K. Kahle, Boston, MA (US); Sattar Khoshkhoo, Boston, MA (US); Alice Lee, Boston, MA (US); Yilan Wang, Boston, MA (US)

(73) Assignees: The Children's Medical Center Corporation, Boston, MA (US); The General Hospital Corporation, Boston, MA (US); The Brigham and Women's Hospital, Inc., Boston, MA (US)

(21) Appl. No.: 19/366,227

(22) Filed: Oct. 22, 2025

Related U.S. Application Data
(63) Continuation of application No. PCT/US2023/085893, filed on Dec. 26, 2023.
(60) Provisional application No. 63/462,890, filed on Apr. 28, 2023.

(51) Int. Cl. A61K 31/519; A61K 31/196; A61K 31/4184; A61K 31/437; A61K 31/4375; A61K 31/44; A61K 31/4439; A61K 31/497; A61K 31/4985; A61K 31/506; A61K 31/517; A61K 31/5377; A61P 25/08; C12Q 1/6883

(52) U.S. Cl. CPC A61K 31/519; A61K 31/196; A61K 31/4184; A61K 31/437; A61K 31/4375; A61K 31/44; A61K 31/4439; A61K 31/497; A61K 31/4985; A61K 31/506; A61K 31/517; A61K 31/5377; A61P 25/08; C12Q 1/6883; C12Q 2600/156

(57) ABSTRACT

The disclosure provides compositions and methods for characterizing or treating a seizure disorder (e.g., focal epilepsy, temporal lobe epilepsy, mesial temporal lobe epilepsy, and related disorders).

### CROSS-REFERENCE TO RELATED APPLICATIONS

The present application is a continuation under 35 U.S.C. § 111(a) of PCT International Patent Application No. PCT/US2023/085893, filed Dec. 26, 2023, designating the United States and published in English, which claims priority to and the benefit of U.S. Provisional Application No. 63/462,890, filed Apr. 28, 2023, which is hereby incorporated by reference in its entirety.

### STATEMENT OF RIGHTS TO INVENTIONS MADE UNDER FEDERALLY SPONSORED RESEARCH

This invention was made with government support under Grant No. NS035129, NS065743, NS109358, NS111029, NS117609, and NS128272, awarded by the National Institutes of Health. The Government has certain rights in the invention.

### BACKGROUND OF THE INVENTION

Epilepsy is a debilitating chronic neurologic condition that affects 1 in 26 people (3-4% lifetime risk). The most common focal epilepsy subtype, mesial temporal lobe epilepsy (MTLE), is often resistant to anti-seizure medications and requires neurosurgical intervention (e.g., anterior temporal lobectomy) in roughly one-third of the patients, with attendant morbidity. While MTLE has been associated with initial precipitating insults such as prolonged febrile seizures and trauma, its etiology is debated and its pathophysiology is poorly understood. With a few exceptions, whole-exome sequencing (WES) and gene-panel sequencing (GPS) studies of blood- and buccal-derived DNA have had minimal success in identifying genetic determinants of focal epilepsies such as MTLE, which typically occur in individuals without a family history of the disease. Accordingly, improved methods for characterizing and treating subjects with MTLE are urgently required.

### SUMMARY OF THE INVENTION

The disclosure provides compositions and methods for characterizing and/or treating mesial temporal lobe epilepsy (MTLE).

In an aspect, the present disclosure provides a method of treating a subject having or having a propensity to develop a seizure disorder. The method involves administering to the subject an agent that inhibits a component of a Ras/Raf/MapK signaling pathway, where the subject is selected by detecting an alteration in the sequence of a polynucleotide encoding a polypeptide that is a component of the Ras/Raf/MapK signalling pathway, where the polynucleotide is present in a biological sample of the subject, thereby treating the subject.

In another aspect, the present disclosure provides a method of treating a subject having or having a propensity to develop a seizure disorder associated with a somatic mutation in a Ras/Raf/MapK signaling pathway. The method involves administering to the subject an agent that inhibits a component of a Ras/Raf/MapK signaling pathway, thereby treating the subject.

In another aspect, the present disclosure provides method of treating a subject having or having a propensity to develop a seizure disorder associated with a somatic mutation in a gene. The gene is PTPN11, FGFR1, KRAS, NF1, BRAF, RASA1, RAF1, RIT1, or CBL. The method involves administering to the subject an agent that inhibits a component of a Ras/Raf/MapK signaling pathway, thereby treating the subject.

In any of the above aspects, or embodiments thereof, the component of the Ras/Raf/MapK signaling pathway is PTPN11, KRAS, SOS1, BRAF, CBL, LZTR1, PIK3CA, or NF1.

In any of the above aspects, or embodiments thereof, the seizure disorder is epilepsy, a focal epilepsy subtype, temporal lobe epilepsy, or mesial temporal lobe epilepsy.

In any of the above aspects, or embodiments thereof, the polynucleotide is genomic DNA, RNA, or cell free DNA.

In any of the above aspects, or embodiments thereof, the biological sample includes cerebrospinal fluid, tissue, plasma, or serum.

In any of the above aspects, or embodiments thereof, the agent is a RAS, RAF, MAPK, BRAF, MEK, RAF or ALK inhibitor.

In any of the above aspects, or embodiments thereof, the agent is AMG 510, MRTX849, sorafenib, vemurafenib, dabrafenib or encorafenib, PLX8394, LY3009120, belvarafenib, LXH254, rigosertib, trametinib, dabrafenib, encorafenib, binimetinib, trametinib and selumetinib, ulixertinib and ONC201, erlotinib, lapatinib, momelotinib, pan-RAF inhibitor tovorafenib, vemurafenib, dabrafenib, MEK162, RAF265, XL281/BMS-908662, sorafenib, ASP-3026, alectinib (ALECENSA), brigatinib (AP26113), ceritinib (ZYKADIA), CEP-28122, CEP-37440, crizotinib (XALKORI), entrectinib (e.g., NMS-E628, RXDX-101), PF-06463922, TSR-011, X-376, X-396, TNO155, RMC-4630, JAB-3068, RLY-1971, ERAS-601, BBP-398, or combinations thereof.

In any of the above aspects, or embodiments thereof, the component of the Ras/Raf/MapK signaling pathway is PTPN11/SHP2. In any of the above aspects, or embodiments thereof, the alteration in PTPN11/SHP2 is D61N, D61G, D61Y, A72V, A72S, A72D, A72T, A72P, E76K, E76V, E76G, E76A, Y297S, F285S, F285C, N308D, N308S, A461T, A461G, P491S, P491H, P498 W, P498L, S502L, S502A, G503V, G503A, G503R, M504V, Q506P, T507K, Q510E, Q510P, and/or Q510H.

In any of the above aspects, or embodiments thereof, the somatic mutation in a Ras/Raf/MapK signaling pathway is a somatic mutation in PTPN11/SHP2. In any of the above aspects, or embodiments thereof, the somatic mutation in PTPN11/SHP2 is D61N, D61G, D61Y, A72V, A72S, A72D, A72T, A72P, E76K, E76V, E76G, E76A, Y297S, F285S, F285C, N308D, N308S, A461T, A461G, P491S, P491H, P498 W, P498L, S502L, S502A, G503V, G503A, G503R, M504V, Q506P, T507K, Q510E, Q510P, and/or Q510H.

In any of the above aspects, or embodiments thereof, the gene is PTPN11. In any of the above aspects, or embodiments thereof, the somatic mutation in PTPN11 is D61N, D61G, D61Y, A72V, A72S, A72D, A72T, A72P, E76K, E76V, E76G, E76A, Y297S, F285S, F285C, N308D, N308S, A461T, A461G, P491S, P491H, P498 W, P498L, S502L, S502A, G503V, G503A, G503R, M504V, Q506P, T507K, Q510E, Q510P, and/or Q510H.

In any of the above aspects, or embodiments thereof, the agent is TNO155, RMC-4630, JAB-3068, RLY-1971, ERAS-601, BBP-398, or combinations thereof.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a polypeptide or fragment thereof, polynucleotide, or small molecule compound. In embodiments, the agent inhibits Ras-MAPK signaling, or inhibits a component of a Ras-MAPK signaling pathway. In embodiments, the agent is AMG 510, MRTX849, sorafenib, vemurafenib, dabrafenib or encorafenib, PLX8394, LY3009120, belvarafenib, LXH254, rigosertib, trametinib, dabrafenib, encorafenib, binimetinib, trametinib and selumetinib, ulixertinib and ONC201, erlotinib, lapatinib, momelotinib, pan-RAF inhibitor tovorafenib, vemurafenib, dabrafenib, MEK162, RAF265, XL281/BMS-908662, sorafenib, ASP-3026, alectinib (ALECENSA), brigatinib (AP26113), ceritinib (ZYKADIA), CEP-28122, CEP-37440, crizotinib, TNO155, RMC-4630, JAB-3068, RLY-1971, ERAS-601, or BBP-398.

By “alteration” is meant a change in the structure, expression levels or activity of a gene or polypeptide as detected by standard art known methods such as those described herein. In the context of expression or activity an alteration may be an increase or decrease. As used herein, an alteration includes a 10% change in expression or activity levels, preferably a 25% change, more preferably a 40% change, and most preferably a 50% or greater change in expression levels. In embodiments, an alteration is a mutation in a gene (e.g., a component of a RAS/RAF/MAPK pathway).

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “deep sequencing” is meant sequencing a region of a polynucleotide tens or hundreds or even thousands of times. In some embodiments, deep sequencing includes next-generation sequencing, high-throughput sequencing and massively parallel sequencing. Deep sequencing involves obtaining large numbers of sequences corresponding to relatively short, targeted regions of a genome. A targeted region can include, for example, an entire gene or a portion of a gene (such as a mutation hotspot), or a regulator of the gene (e.g., a promoter or enhancer). In some embodiments, many thousands of clonal sequences are obtained from a short, targeted segment allowing identification and quantitation of somatic single nucleotide variants. In some embodiments, a particular region of a polynucleotide is sequenced for example 100, 250, 500, 1,000, 2,500, 5,000, 7,500, 10,000, 25,000, 50,000, 100,000, 250,000, 500,000, 750,000, or even 1, 5, or 10, 25, 50, 75, or 100 million times.

By “detect,” is meant any method for identifying the presence, absence, or amount of a single nucleotide variation.

By “disease” is meant any condition or disorder that damages or interferes with the normal function of a cell, tissue, or organ. Examples of diseases include, without limitation, diseases and disorders associated with undesirable electrical activity in a brain region, including epilepsy, temporal lobe epilepsy, medial temporal lobe epilepsy, and other related seizure disorders.

By “corresponds” is meant comprising at least a fragment of a double-stranded gene, such that a strand of the double-stranded inhibitory nucleic acid molecule is capable of binding to a complementary strand of the gene.

By “decreases” is meant a reduction by at least about 5% relative to a reference level. A decrease may be by 5%, 10%, 15%, 20%, 25% or 50%, or even by as much as 75%, 85%, 95% or more and any intervening percentages.

“Detect” refers to identifying the presence, absence or amount of the analyte to be detected.

The term “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In an embodiment, an effective amount if the amount required to reduce the frequency, severity, or number of seizures in a subject.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “high-throughput sequencing” is meant a sequencing technique that allows for large amounts of nucleic acids to be sequenced.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free, to varying degrees, from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “gene” refers to a nucleic acid molecule or portion of a nucleic acid molecule comprising a sequence that encodes a protein. It is understood in the art that a gene also comprises non-coding sequences, such as 5′ and 3′ flanking sequences (such as promoters, enhancers, repressors, and other regulatory sequences) as well as introns.

As used herein, the term “mutation” is meant to include any genetic alteration. Genetic alterations may occur in a protein coding or in a regulatory sequence. Exemplary mutations include point mutations and small insertion/deletion mutations (e.g., 1-50-bp insertion or deletion mutation). In embodiments, mutations can lead to changes in the structure of an encoded protein or to a decrease or complete loss in its expression.

As used herein, the term “nucleic acid molecule” refers to a polymeric form of nucleotides. Exemplary polynucleotides include ribonucleotides or deoxyribonucleotides (e.g., RNA, DNA, and combinations or analogs thereof.

“Primer set” means a set of oligonucleotides that may be used for DNA amplification, for example, PCR. A primer set would consist of at least 2, 4, 6, 8, 10, 12, 14, 16, 18, 20, 30, 40, 50, 60, 80, 100, 200, 250, 300, 400, 500, 600, or more primers.

By “reference” is meant a standard or control condition. In one embodiment, the sequence of a gene in a subject having or having a propensity to develop epilepsy, MTLE, TLE, or another seizure disorder is compared to a reference sequence, such as the sequence of a healthy control subject.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “MAP Kinase Pathway” is meant a conserved signal transduction pathway in which activated Ras induces a kinase cascade that activates MAP kinase. Proteins within the MAP kinase pathway include, for example, ALK, RAF, EGFR, RAS, and MEK. The MAP Kinase Pathway is described, for example, by Lodish et al., Molecular Cell Biology, 4^{th} edition, New York; W.Hl. Freeman, 2000, at section 20.5 Map Kinase Pathways, which is incorporated herein by reference.

By “MAP Kinase Pathway Inhibitor” is meant any agent that inhibits the activity of the Map kinase pathway. Exemplary MAPK pathway inhibitors include ALK inhibitors, MEK inhibitors, BRAF inhibitors, SHP2 inhibitors, or EGFR inhibitors, as specified herein.

By “ALK inhibitor” is meant an agent that reduces or eliminate a biological function or activity of an ALK polypeptide (e.g., anaplastic lymphoma kinase). Exemplary biological activities or functions of an ALK polypeptide include receptor tyrosine protein kinase activity. Examples of an ALK inhibitor include, without limitation ASP-3026, alectinib (ALECENSA), brigatinib (AP26113), ceritinib (ZYKADIA), CEP-28122, CEP-37440, crizotinib (XALKORI), entrectinib (e.g., NMS-E628, RXDX-101), PF-06463922, TSR-011, X-376 and X-396.

By “marker” is meant any protein or polynucleotide having an alteration in expression level or activity that is associated with a disease or disorder.

By “portion” is meant a fragment of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 6, 7, 8, 9, 10, 11, 12, 13, 14, 15, 16, 17, 18, 19, 20, or 21 nucleotides.

By “positioned for expression” is meant that the polynucleotide of the disclosure (e.g., a DNA molecule) is positioned adjacent to a DNA sequence that directs transcription and translation of the sequence (i.e., facilitates the production of, for example, a recombinant microRNA molecule described herein).

By “RNA-seq” is meant RNA sequencing for detecting and quantifying messenger RNA molecules (mRNA) in a biological sample, which, for example, may be used to study cellular responses. A related term, “scRNA-seq” is single-cell RNA sequencing, which may be, for example, a droplet-based single-cell RNA-seq or “Drop-seq,” that is a sequencing technology for analyzing RNA expression in at least hundreds of thousands of individual cells in embodiments of the disclosure, but may alternatively use any other high-throughput sequencing platform.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100.mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.10% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

As used herein, the term “sequencing” and its variants comprise obtaining sequence information from a nucleic acid strand, typically by determining the identity of at least some nucleotides (including their nucleobase components) within the nucleic acid molecule. The term sequencing may also refer to determining the order of nucleotides (base sequences) in a nucleic acid sample, e.g., DNA or RNA. Many techniques are available such as Sanger sequencing and high-throughput sequencing technologies (also known as next-generation sequencing technologies) such as the GS FLX platform offered by Roche Applied Science, based on pyro sequencing. In an embodiment sequencing encompasses, for example, deep whole exome sequencing (WES) and gene panel sequencing.

The terms “somatic mutation,” and “somatic single-nucleotide variation,” refer to an alteration in a polynucleotide sequence of a somatic cell that may or may not be shared by other cells on the basis of their derivation from a common progenitor cell.

By “somatic mutational burden” is meant a measure of the number of somatic mutations within a cell.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal (e.g., a bovine, equine, canine, ovine, feline, rodent, or primate).

The term “variant” is used herein to refer to a change or alteration in sequence relative to a reference sequence at a particular locus. In embodiments the alteration or variant is a nucleotide base substitution, deletion, or insertion in coding or non-coding regions.

The term “single nucleotide variant,” or “single nucleotide variation,” (SNV) refers to a single nucleotide alteration at a particular site. SNVs occur without any limitations of frequency and may arise in somatic cells, e.g., a “somatic single-nucleotide variant (sSNV).” In various embodiments, the sSNV is identified by the presence of a complementary nucleotide (G-C; A-T) on the opposite strand.

The term “single nucleotide polymorphism” (SNP) refers to a single nucleotide alteration at a particular site that occurs in at least about 1% of the general population of a species. In the human genome, single nucleotide polymorphisms occur about once in every 300 nucleotide base pairs. SNPs may or may not be located within genes and may or may not affect gene expression or protein function.

By “unique molecular identifier” or “UMI” is meant a short nucleic acid sequence that is identifiable in, for example, high-throughput sequencing techniques. In embodiments, the sequencing method is single-cell RNA-seq.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1. provides a diagram of Somatic Variants Activating Ras/Raf/MAPK Pathway Genes in MTLE. Simplified diagram of the Ras/Raf/MAPK signaling pathway, the pathogenic variants discovered in the MTLE cohort, and their corresponding proteins. RTK is Receptor Tyrosine Kinase.

FIGS. 2A-2B. provide diagrams and graphs showing enrichment of Ras/Raf/MAPK Pathway Variants in the Temporal Lobe and in MTLE. FIG. 2A shows a retrospective review of Ras/Raf/MAPK pathway variants and PI3K/Akt/mTOR pathway variants in the lesional focal epilepsy literature. Circle diameters represent normalized case counts in each brain region and the associated bar plots on the right depict the absolute number of cases. FIG. 2B shows KEGG (Kyoto Encyclopedia of Genes and Genomes) pathway enrichment analysis on the pathogenicity enriched variants in the MTLE cohort (p-value<0.001 and adjusted p-value<0.05 after correction for multiple hypotheses testing). The dashed red line represents the adjusted p-value of 0.05.

FIGS. 3A-3G. provides pictures and graphs showing mechanisms of Ras/Raf/MAPK Overactivity in MTLE-Associated Mutant Shp2 Proteins. FIG. 3A shows an immunoblot of the indicated proteins in HEK293T cells transiently expressing Shp2wt and Shp2mut. FIG. 3B shows representative pictures from live imaging microscopy of Shp2wt-mEGFP and Shp2mut-mEGFP proteins, transiently expressed in KYSE520 cells. The fluorescent puncta (marked by white arrowheads) are quantified FIG. 3C (mean±SEM, p<0.001 mut vs wt). FIG. 3D shows representative images of FRAP using the transiently expressed Shp2G503R-mEGFP protein in KYSE520 cells. The rate of fluorescence recovery is quantified in FIG. 3E (mean±SEM, n=3 experiments). FIG. 3F shows representative images of 8 μM Shp2 wt and Shp2mut protein droplet formation in the presence of 10% (w/v) PEG3350 in vitro. Droplet formation is quantified by solution turbidity of OD600 in FIG. 3G (mean±SEM, p<0.001 mut vs wt.

FIGS. 4A-4I. provides pictures showing evidence of Erk1/2 Overactivation in MTLE Tissue Harboring Ras/Raf/MAPK Variants Each column shows histopathologic images from one sample. FIGS. 4A, 4F) A long-term epilepsy associated tumor (LEAT) with areas of hypercellularity corresponding to the tumor (FIG. 4A1) intermixed with normal brain tissue (FIG. 4A2). pErk1/2 staining of the corresponding regions is seen in FIGS. 4F, 4F1-2. FIGS. 4B-4E, 4G-4H) Representative MTS-only samples with neuronal dropout in the Comu Ammonis (CA) region of the hippocampus (FIGS. 4B-4E) but uniform distribution of glial cells across the same region (FIG. 4G). The black arrows demarcate CA neuronal loss. Erk1/2 phosphorylation colocalizes to regions with highest degree of neuronal loss and sclerosis (FIGS. 4H, 4I). The white arrowheads point to cells with increased pErk1/2 staining, mostly represented by apparent glia as highlighted in the panel I inset. The black arrowheads point to neurons which have relatively low pErk1/2 staining as highlighted in FIG. 4H1 inset. The higher resolution images were obtained at 200-400× and the lower resolution images were obtained at 40×.

FIG. 5 provides a schematic representation of the RAS-MAPK signaling pathway with various inhibitors useful in the treatment of seizure disorders (e.g., TLE, MTLE), which was taken from Mlakar et al., J. Exp. Clin. Cancer Res. 40:189, 2021, which is incorporated herein by reference in its entirety.

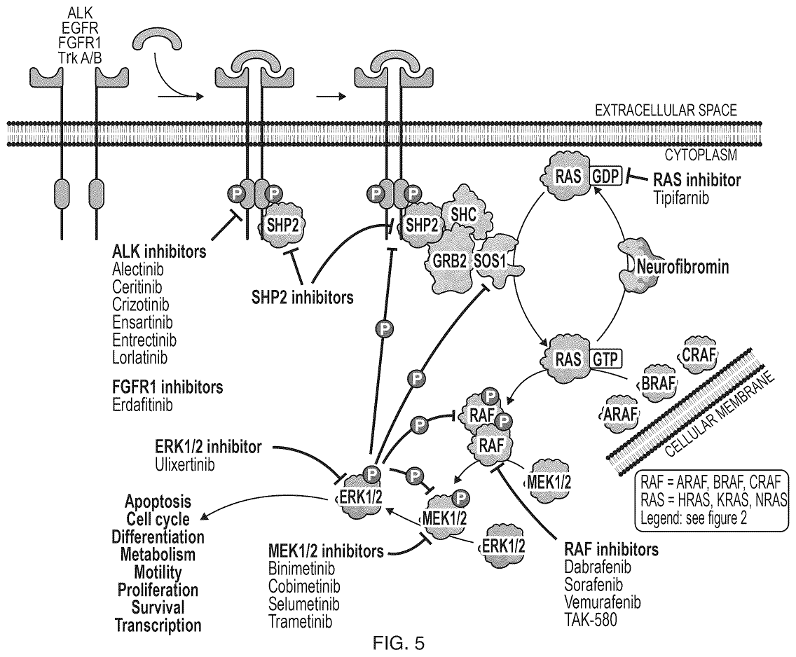

FIGS. 6A-6B provide graphs showing mean sequencing depth for MTLE and neurotypical control samples. FIG. 6A shows that >500× mean sequencing depth was achieved for most MTLE hippocampal samples. MTLE temporal neocortical samples were also sequenced deeply, although for paired variant calling, they were downsampled to 50× to be used as germline controls for somatic variant detection, as described in the methods. FIG. 6B shows the sequencing depth for the neurotypical control samples. For any analyses comparing MTLE and control samples, the MTLE bam files were downsampled to the same average mean depth as the controls, 284×, as described in the methods. Boxes show the lower and upper quartiles and the line inside the box is median. Whiskers indicate lower and upper extremes of the distribution. Each point represents an individual sample.

FIGS. 7A-7B provides pie charts showing the two-year epilepsy surgery outcome. The two-year epilepsy surgery outcome for study participants is summarized in pie chart format based on both ILAE and Engel classification systems. Only participants with sufficient clinical follow up data at the 2-year postsurgical timepoint are shown here (n=67).

FIGS. 8A-8P. provides pictures showing MTLE cases with activating Ras/Raf/MAPK variants and MTS-only pathology. Radiographic and histopathologic findings in patients with MTS-only pathology are shown in FIGS. 8A-8P. Each column exhibits data for one patient. FIGS. 8A-8D) Representative T2/FLAIR coronal MRI images show hippocampal atrophy (FIGS. 8A-8D) and T2/FLAIR signal hyperintensity (FIGS. 8B-8D), which are consistent with MTS. Expansile or contrast-enhancing lesions are not seen. FIGS. 8E-8L) Representative histopathological images from the surgically resected hippocampi (demarcated by the dashed white circle in the first row). FIGS. 8H&8E and NeuN staining show neuronal loss in the Cornu Ammonis (CA) region of the hippocampus (black arrows). FIGS. 8M-8P) Background-level CD34 staining of blood vessels with no evidence of a glioneuronal tumor. The higher resolution images were obtained at 100-200× and the lower resolution images were obtained at 40×.

FIGS. 9A-9N provides pictures showing MTLE cases with activating Ras/Raf/MAPK variants and MTS+ pathology. Each column in this figure corresponds to one patient. In the first two rows, representative FLAIR/T1 axial and T2/STIR coronal MRI images highlight the main radiographic abnormality in each case (demarcated by the dashed white circle in the first row). In mcdbose315, FIG. 9C, the dysplasia starts in the anterior medial temporal region and extends posteriorly to the temporal/occipital junction, indicating an early gestational origin. Rows 3 and 4 show some of the key histopathologic findings in each patient. Areas of hypercellularity shown in FIGS. 9E and 91 are consistent with a low-grade tumor. Focal microcolumnar arrangement of cortical neurons in FIG. 9F and SMI31+atypical neurons (black arrow) in FIG. 9J are consistent with a diagnosis of FCD type 1A. FIG. 9G shows indistinct cortical layers which in conjunction with hamartias (white arrowhead) in FIG. 9K and dysplastic neurons (black arrowhead) in panel L are supportive of a diagnosis of FCD type 2A. Mild glial and neuronal atypia in FIG. 9H (highlighted in the inlet) and clusters of neurons with crowding that lack lamination in FIG. 9M, are consistent with a diagnosis of a glioneuronal tumor. FIG. 9N highlights rare perikaryal staining of atypical neurons with SMI31. The higher resolution images were obtained at 100-200× and the lower resolution images were obtained at 40×.

FIGS. 10A-10F provide graphs showing expression of Ras/Raf/MAPK genes with MTLE-associated somatic variants in the adult human brain. Published single cell RNA sequencing data from MTLE hippocampal tissue was reanalyzed (FIGS. 6-9) to define the major cell clusters (FIG. 10A) and to quantify the RNA expression levels for Ras/Raf/MAPK Genes with MTLE-associate Somatic Variants (FIGS. 10B-10F). All the Ras/Raf/MAPK genes with pathogenic somatic variants, PTPN11, KRAS, SOS1, NF1, and BRAF, are expressed in both neurons and glia in the adult human hippocampus from patients with MTLE. The scale bar values represent log-normalized RNA expression levels.

FIG. 11 provides a graph showing frequent, highly recurring, PTPN11/RAS/RAF/MAPK mutations in mTLE. Shown are examples of frequent mutation sin PTPN11/SHP2 associated with mTLE. All shown mutations are missense mutations. The vertical axis represents the absolute number of patients showing each allele.

FIGS. 12A-12B provide graphs showing that PTPN11/RAS/RAF/MAPK mutations show clonal selection. FIG. 12A shows clonal selection for RAS/RAF/MAPK variants in the TLE brain. In this graph, the lower dots represent a normal brain, and the upper dots represent the TLE brain. FIG. 12B shows that 50/50 mixing of isogenic wild-type and mutant (PTPN11 G503R) iPSC cell lines shows that mutant cells had a distinct selection advantage.

### DETAILED DESCRIPTION OF THE INVENTION

The invention features compositions and methods for characterizing and treating seizure disorders.

The disclosure is based, at least in part, on the discovery that hippocampal somatic variants activating Ras-MAPK signaling contribute to the pathogenesis of sporadic, drug-resistant TLE in a significant proportion of patients. These findings may provide a novel genetic mechanism and highlight new therapeutic targets for this most common form of focal epilepsy that does not have any disease-modifying treatments available right now. Since the Ras-MAPK pathway is one of the most common targets for cancer therapies, with several FDA approved drugs already in regular clinical use, these findings should have immediate clinical impact.

### Epilepsy

Epilepsy is a serious chronic neurologic condition that affects over 50 million people worldwide. Mesial temporal lobe epilepsy (TLE)—the most common focal epilepsy—is treatment-refractory in roughly one-third of the patients, resulting in significant neurologic and psychiatric morbidity. Most TLE patients do not have inherited or de novo germline genetic mutations and therefore genetic factors were previously not thought to play a major role in this condition. However, the contribution of post-zygotic (i.e., somatic) mutations to TLE had been unknown prior to the results provided herein. Using a deep whole-exome sequencing (>500×) approach, we analyzed the DNA derived from hippocampal tissue of neurosurgically-treated TLE patients and age- and sex-matched neurotypical controls.

We found that pathogenic somatic mutations are enriched in the hippocampus (seizure focus) in TLE relative to the unaffected temporal neocortex, and absent in neurotypical controls. Almost all the pathogenic somatic mutations in TLE are predicted to activate Ras-MAPK signaling. These results indicate that activating Ras-MAPK pathway mutations give rise to a spectrum of temporal lobe lesions (mesial temporal sclerosis, focal cortical dysplasia, and low-grade epilepsy-associated tumors), all causing focal, drug-resistant TLE. Somatic Ras-MAPK mutations appear to have a special propensity for causing focal epilepsy in the temporal lobe, contrary to extra-temporal focal epilepsies which are frequently associated with PI3K-mTOR pathway somatic mutations. TLE-associated somatic mutations in PTPN11 appear to activate Ras-MAPK signaling through a dominant gain-of-function mechanism mediated by abnormal liquid-liquid phase separation.

Since cell loss is a prominent feature of mesial temporal sclerosis in TLE, pathogenic variants in the hippocampus may be present at allele fractions<10%, suggesting that the burden of pathogenic somatic variants in TLE could be much higher than what we discovered with whole-exome sequencing. To pursue this find, we designed a gene-panel with duplex sequencing technology to facilitate the detection of ultra-rare somatic mutations in the hippocampus in TLE. Using duplex- and UMI-resolved sequencing data (>1000×) we found that pathogenic somatic Ras-MAPK variants, in particular variants in PTPN11, FGFR1, KRAS, NF1, BRAF, RASA1, RAF1, RIT1, and CBL, are present in >30% of surgically resected hippocampal tissue from drug-resistant TLE cases.

The majority of TLE-associated pathogenic variants are in PTPN11 and mostly clustered in two hot-spots across the gene. The TLE-associated pathogenic variants are enriched in the hippocampus compared to the unaffected temporal neocortical tissue. There is a statistically significant increased burden of pathogenic variants in TLE compared to controls. In the TLE cohort, there is a statistically significant burden of pathogenic variants in Ras-MAPK genes relative to PI3K-mTOR and developmental and epileptic encephalopathy genes.

In summary, for the first time, we established that hippocampal somatic variants activating Ras-MAPK signaling contributed to the pathogenesis of sporadic, drug-resistant TLE in a significant proportion of patients. These findings provide a novel genetic mechanism and highlight new therapeutic targets for this most common form of focal epilepsy that does not have any disease-modifying treatments available right now. Accordingly, drugs targeting the Ras-MAPK pathway should be useful for the treatment of TLE and other related seizure disorders.

### Pharmacologic Inhibition of RAS/RAF/MAPK

Current methods for treating treatment refractory subjects with TLE typically involve surgical ablation, which may have adverse effects on behavior and cognition. Accordingly, there is an urgent need for pharmacologic intervention for such subject. The present disclosure provides agents that have been shown to inhibit the Ras/Raf/MAPK signaling pathway in subject having a seizure disorder associated with a somatic mutation that activates Ras-MAPK signaling. Such agents include, but are not limited to AMG 510, MRTX849, sorafenib, vemurafenib, dabrafenib or encorafenib, PLX8394, LY3009120, belvarafenib and LXH254, rigosertib, trametinib, dabrafenib, encorafenib, binimetinib, trametinib and selumetinib, ulixertinib and ONC201, erlotinib, lapatinib, and momelotinib, pan-RAF inhibitor tovorafenib. Other inhibitors useful in connection with TLE or MTLE include BRAF, MEK, RAF and ALK inhibitors.

Examples of a BRAF inhibitor include, without limitation, vemurafenib and dabrafenib. Examples of a MEK inhibitor include, without limitation, trametinib, selumetinib, and MEK162. Examples of a SHP2 inhibitor include, without limitation, TNO155, RMC-4630, JAB-3068, RLY-1971, ERAS-601, and BBP-398. In some embodiments, the RAF inhibitor is RAF265, XL281/BMS-908662, or sorafenib. In some embodiments, the ALK inhibitor can be ASP-3026, alectinib (ALECENSA), brigatinib (AP26113), ceritinib (ZYKADIA), CEP-28122, CEP-37440, crizotinib (XALKORI), entrectinib (e.g., NMS-E628, RXDX-101), PF-06463922, TSR-011, X-376 and X-396.

Other inhibitors useful in the methods described herein are provided in the following references: J. Canon, K. Rex, A. Y. Saiki, C. Mohr, K. Cooke, D. Bagal, K. Gaida, T. Holt, C. G. Knutson, N. Koppada, et al. The clinical KRAS(G12C) inhibitor AMG 510 drives anti-tumor immunity, Nature, 575 (2019), pp. 217-223; Hallin, L. D. Engstrom, L. Hargis, A. Calinisan, R. Aranda, D. M. Briere, N. Sudhakar, V. Bowcut, B. R. Baer, J. A. Ballard, et al. The KRASG12C inhibitor, MRTX849, provides insight towards therapeutic susceptibility of KRAS mutant cancers in mouse models and patients, Cancer Discov., 10 (2020), pp. 54-71; Savoia, P. Fava, F. Casoni, O. Cremona, Targeting the ERK signaling pathway in melanoma, Int. J. Mol. Sci., 20 (2019), p. E1483; Yao, Y. Gao, W. Su, R. Yaeger, J. Tao, N. Na, Y. Zhang, C. Zhang, A. Rymar, A. Tao, et al. RAF inhibitor PLX8394 selectively disrupts BRAF dimers and RAS-independent BRAF-mutant-driven signaling, Nat. Med., 25 (2019), pp. 284-291; Zhang, W. Spevak, Y. Zhang, E. A. Burton, Y. Ma, G. Habets, J. Zhang, J. Lin, T. Ewing, B. Matusow, et al. RAF inhibitors that evade paradoxical MAPK pathway activation, Nature, 526 (2015), pp. 583-586; Durrant, D. K. Morrison, Targeting the Raf kinases in human cancer: the Raf dimer dilemma, Br. J. Cancer, 118 (2018), pp. 3-8.

### Types of Biological Samples

Compositions and methods described herein involve identifying somatic mutations from nucleic acids taken from a biological sample of a subject. The biological samples are generally derived from the subject in the form of a bodily fluid (e.g., blood, cerebrospinal fluid, phlegm, saliva, sputum, semen, vaginal secretion, or urine) or tissue sample (e.g. a cheek swab, scraping, or tissue sample obtained by biopsy). In some preferred embodiments, the fluid sample is a blood sample or cerebrospinal fluid sample. The cerebrospinal fluid sample can be collected through a procedure called a spinal tap, also known as a lumbar puncture.

### Target Sequence Analysis

Analyzing the target sequence or fragment thereof that hybridizes to one or more of the selected probes may involve sequencing, FACS, qPCR, RT-PCR, a genotyping array, and/or a NanoString assay (see, e.g., Malkov, et al. “Multiplexed measurements of gene signatures in different analytes using the Nanostring nCounter™ Assay System”, BMC Research Notes, 2: Article No: 80 (2009)), or any of various other techniques known to one of skill in the art. Various characterization methods may be used and are described as follows.

RNA sequencing (RNA-Seq) is a powerful tool for transcriptome profiling. In embodiments, to mitigate sequence-dependent bias resulting from amplification complications to allow truly digital RNA-Seq, a set of barcode sequences can be used to ensure that every cDNA molecule prepared from an mRNA sample is uniquely labeled by random attachment of barcode sequences to both ends (see, e.g., Shiroguchi K, et al. Proc Natl Acad Sci USA. 2012 Jan. 24; 109(4):1347-52). After PCR, paired-end deep sequencing can be applied to read the two barcodes and cDNA sequences. Rather than counting the number of reads, RNA abundance can be measured based on the number of unique barcode sequences observed for a given cDNA sequence. The barcodes may be optimized to be unambiguously identifiable. This method is a representative example of how to quantify a whole transcriptome from a sample.

Library preparation may involve an amplification step. Amplification may involve thermocycling or isothermal amplification (such as through the methods RPA or LAMP). Cross-linking may involve overlap-extension PCR or use of ligase to associate multiple amplification products with each other. Amplification can refer to any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dyes suitable for this application include, as non-limiting examples, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are taught, for example, in U.S. Pat. No. 5,210,015.

Sequencing may be performed on any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference). See also Metzker Nature Review Genetics 11, 31-46 (2010).

The sequencing of a polynucleotide can be carried out using any suitable commercially available sequencing technology. In another embodiment, the sequencing of a polynucleotide is carried out using chain termination method of DNA sequencing (e.g., Sanger sequencing). In yet another embodiment, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLiD sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing.

Polynucleotides may be characterized and/or enriched by means of a biochip (also known as a microarray) containing hybrid capture probes of the present invention. Biochips generally comprise solid substrates and have a generally planar surface, to which a capture reagent (also called an adsorbent or affinity reagent) is attached. The capture reagent can be a hybrid capture probe(s) or a binding member. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. Such solid supports are suitable for use as solid supports generally in embodiments of the present invention. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Nucleic Acid Amplification

In some embodiments, nucleic acid molecules obtained from a biological sample derived from a subject are amplified. Nucleic acid amplification involves producing one or more copies of a nucleic acid molecule. An amplification product may be RNA or DNA, and may include a complementary strand to the expressed target sequence. RNA amplification products can be produced initially through reverse transcription (e.g., with reverse transcriptase) to generate cDNA and then optionally from further amplification reactions. The amplification product may include all or a portion of a target sequence, and may optionally be labeled. A variety of amplification methods are suitable for use in the methods described herein, including polymerase-based methods and ligation-based methods. One exemplary amplification technique is the polymerase chain reaction (PCR).

The first cycle of amplification in polymerase-based methods (e.g., PCR) typically involves a primer extension product complementary to the template strand. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR. The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used. Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

In some embodiments, a multiple displacement amplification (MDA) reaction is performed to amplify one or more targets of interest. In some embodiments, the targets include proliferation-related genes, including any one or more of the genes listed in Table 1. The MDA reaction can be performed in a 20 μl total reaction volume by addition of an MDA master mix (2 μl 10× Phi29 reaction buffer (Epicentre), 8.4 μl H₂O, 4 μl 10 mM dNTP, 1 μl 1 mM random hexamer (5′ dNdNdNdN*dN*dN-3′ [where*=thiophosophate linkage])(IDT or Thermo-Fisher), 0.4 μl repliPHI polymerase (40U) (Epicentre)). MDA was performed at 30° C. for 16 hours.

### Primers

Primers based on the nucleotide sequences of a target polynucleotide may be designed for use in amplification of the target sequences. For use in amplification reactions, such as PCR, a pair of primers is used. The exact composition of the primer sequences is not critical to the invention, but for most applications the primers hybridize to specific sequences under stringent conditions, particularly under conditions of high stringency, as known in the art. The pairs of primers are typically positioned to generate an amplification product. In embodiments, an amplification product comprises at least about 25, 50, 75 or at least about 100 nucleotides. Algorithms for the selection of primer sequences are generally known, and are available in commercial software packages. Primers for use in the methods described herein may be used in standard quantitative or qualitative PCR-based assays to assess transcript expression levels of RNAs defined by a probe set. Alternatively, primers are used in combination with probes, such as molecular beacons in amplifications using real-time PCR. In some embodiments, the primers are designed to hybridize to sequences flanking one or more proliferation-related genes. In some embodiments, the primers are designed to hybridize to sequences flanking one or more tumor suppressor genes. In some embodiments, the primers are designed to hybridize to sequences flanking at least a portion of one or more genes listed herein (e.g., (PTPN11, KRAS, SOS1, BRAF)

Primers and probes useful in the methods described herein comprise oligonucleotides containing modified backbones or non-natural internucleoside linkages. As is known in the art, a nucleoside is a base-sugar combination and a nucleotide is a nucleoside that also includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Oligonucleotides having modified backbones include those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary polynucleotide primers having modified oligonucleotide backbones include, for example, those with one or more modified internucleotide linkages that are phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′ amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Other modifications may be made at other positions on the polynucleotide probes or primers, particularly the 3′ position of the sugar on the 3′ terminal nucleotide or in 2′-5′ linked oligonucleotides and the 5′ position of 5′ terminal nucleotide. Polynucleotide probes or primers may also comprise sugar mimetics, such as cyclobutyl moieties in place of the pentofuranosyl sugar.

Polynucleotide primers may also include modifications or substitutions to the nucleobase. As used herein, “unmodified” or “natural” nucleobases include the purine bases adenine (A) and guanine (G), and the pyrimidine bases thymine (T), cytosine (C) and uracil (U).

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. L, ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the polynucleotide probes of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability.

One skilled in the art recognizes that it is not necessary for all positions in a given polynucleotide probe or primer to be uniformly modified. The present disclosure, therefore, contemplates the incorporation of more than one of the aforementioned modifications into a single polynucleotide probe or even at a single nucleoside within the probe or primer.

One skilled in the art also appreciates that the nucleotide sequence of the entire length of the polynucleotide probe or primer does not need to be derived from the target sequence. Thus, for example, the polynucleotide probe may comprise nucleotide sequences at the 5′ and/or 3′ termini that are not derived from the target sequences. Nucleotide sequences that are not derived from the nucleotide sequence of the target sequence may provide additional functionality to the polynucleotide probe. For example, they may provide a restriction enzyme recognition sequence or a “tag” that facilitates detection, isolation, purification or immobilization onto a solid support. In some embodiments, they may provide a UMI. Alternatively, the additional nucleotides may provide a self-complementary sequence that allows the primer/probe to adopt a hairpin configuration. Such configurations are necessary for certain probes, for example, molecular beacon and Scorpion probes, which can be used in solution hybridization techniques.

### Single Cell Whole Genome Sequencing

Whole genome sequencing (also known as “WGS”) is a process that determines the DNA sequence of an organism's genome. In various embodiments, the genome of a single cell is sequenced, including from a nucleus isolated from the cell. Methods of isolating single cells and nuclei of cells are known in the art. For single cell WGS, whole genome amplification is used to construct a library. A common strategy used for WGS is shotgun sequencing, in which DNA is broken up randomly into numerous small segments, which are sequenced. Sequence data obtained from one sequencing reaction is termed a “read.” The reads can be assembled together based on sequence overlap. The genome sequence is obtained by assembling the reads into a reconstructed sequence.

Sequencing of library fragments can be determined by any known method for DNA sequencing. However, high throughput sequencing methods are generally preferred. In one embodiment, the sequencing of a DNA fragment is carried out using commercially available sequencing technology, e.g., SBS (sequencing by synthesis) by Illumina. In yet another embodiment, the sequencing of the DNA fragment is carried out using one of the commercially available next-generation sequencing technologies, including SMRT (single-molecule real-time) sequencing from Pacific Biosciences, Ion Torrent™ sequencing from ThermoFisher Scientific, Pyrosequencing (454) from Roche, and SOLiD® technology from Applied Biosystems. Any appropriate sequencing technology may be chosen for sequencing.

All sequencing libraries contain finite pools of distinct DNA fragments. In a sequencing experiment only some of these fragments are sampled. As used herein, the term “coverage” refers to the percentage of genome covered by reads. Coverage also refers to, in shotgun sequencing, the average number of reads representing a given nucleotide in the reconstructed sequence. Biases in sample preparation, sequencing, and genomic alignment and assembly can result in regions of the genome that lack coverage (that is, gaps) and in regions with much higher coverage than theoretically expected. The term depth may also be used to describe how much of the complexity in a sequencing library has been sampled.

### Whole-Genome Sequencing

In some embodiments, DNA libraries are prepared as previously described G. D.

Evrony et al., Cell 151, 483-496 (2012); G. D. Evrony et al., Neuron 85, 49-59 (2015), which are incorporated by reference. In some embodiments, 500ng of amplified DNA from the isolated nuclei described above are sheared on a Covaris E210 focused ultra-sonicator. Paired-end barcoded whole genome sequencing (WGS) libraries can then be prepared with a NEXTflex DNA sequencing kit using 8 cycles of PCR amplification. Paired-end sequencing (100 bp×2 or 101 bp×2) can be performed on Illumina HiSeq 2000 sequencers at the Harvard Biopolymers Facility (Harvard Medical School, Boston MA) and Axeq (Seoul, South Korea).

In some embodiments, identification of somatic mutations involves high average read depth, such that a low frequency mutation is distinguished from an error as the number of correct reads outnumbers any individual errors that may occur, rendering them statistically irrelevant. The sequencing depth typically ranges from 80× to up to thousands, or even millions-fold coverage (e.g., 100, 1,000, 10,000, 20,000, 50,000, 100,000, 250,000, 500,000, 1,000,000, 250,000,000). In some embodiments, targeted DNA sequencing is to a coverage of about or at least about 10×, 20×, 30×, 40×, 50×, 60×, 70×, 80×, 90×, 100×, 200×, 500×, 1000×, 2000×, or more, where a sequencing coverage of 0.01 indicates that a DNA sample has been sequenced such that the amount of DNA sequenced is equivalent in size to about 1% of the corresponding amplicon from which the DNA sample is derived. In embodiments, the sequencing is to a coverage of no more than about 0.001, 0.01, 0.05, 0.1, 0.2, 0.3, 0.4, 0.5, 0.75, 1, 2, 3, 4, 5, 6, 7, 8, 9, 10, 20, 30, 40, 50, 60, 70, 80, 90, or 100×.

### Whole Transcriptome Sequencing

RNA sequencing (RNA-Seq) is a powerful tool for whole transcriptome profiling. In some embodiments, RNA sequencing is used to identify expressed somatic mutations at the whole-transcriptome scale. RNA sequencing can be performed using methods known in the art. In brief, RNA can be extracted form nucleic prepared as described above. The RNA can be converted into complementary cDNA using a reverse transcriptase enzyme. The cDNA can then be processed for sequencing. To mitigate sequence-dependent bias resulting from amplification complications, a set of unique molecular marker identification sequences can be used to ensure that every cDNA molecule prepared from a nucleic is uniquely labeled. In other embodiments, a molecular barcode is used (see, e.g., Shiroguchi K, et al. Proc Natl Acad Sci USA. 2012 Jan. 24; 109(4):1347-52). After PCR, paired-end deep sequencing can be applied. Rather than counting the number of reads, RNA abundance can be measured based on the number of unique sequences observed for a given cDNA sequence. The barcodes may be optimized to be unambiguously identifiable.

### Compositions and Methods for Assessing Seizure Associated Variants

In some aspects, this disclosure provides oligonucleotides that specifically hybridize to a target gene (e.g., PTPN11, KRAS, SOS1, BRAF, or a gene encoding a component of the Ras/Raf/MAP kinase pathway) comprising one or more somatic mutations, wherein the nucleic acid molecule is derived from a subject having or having a propensity to develop TLE or another seizure disorder as compared to one or more age-matched subjects without a seizure disorder. In some embodiments, the oligonucleotides hybridize to a portion of one or more target genes selected from the group consisting of PTPN11, KRAS, SOS1, BRAF, or a gene encoding a component of the Ras/Raf/MAP kinase pathway. In some embodiments, the oligonucleotide hybridizes to a gene at a genomic position that is between 0-100 base pairs upstream of the target gene. In some embodiments, the nucleic acid probe comprises a sequence that hybridizes to the nucleic acid encoding the gene at a genomic position that is between 0-100 base pairs downstream of the target gene. In some embodiments, the oligonucleotides comprise a pair of oligonucleotides useful in the amplification of a target gene. In other embodiments, the oligonucleotides are primers useful for sequencing a target gene. In still other embodiments, the oligonucleotides comprise one or more probes useful for characterizing the presence of absence of an alteration in a target polynucleotide. The primers and probes may comprise RNA, DNA, or a mixture thereof. In some embodiments, the primers and probes comprise at least about 5, 10, 15, 20, 25, 30, 35, 40, 45, 50, 55, 60, 65, 70, 75, 80, 85, 90, 95, or 100 nucleobases. In some embodiments, the primers and probes comprise a length of between 15-25 base pairs. In some embodiments, the primers and probes comprise one or more modified nucleotides. The modified nucleotides may comprise chemistries that prevent degradation or facilitate detection.

In some embodiments, the nucleic acid probes are linked to a capture moiety. The capture moiety can comprise any material or agent (e.g., affinity tag) that allows the probes to be separated from non-target nucleic acids. In some embodiments, the capture moiety is a solid substrate. In some embodiments, the solid substrate is a bead. In some embodiments, the bead is a magnetic bead. In some embodiments, the solid substrate is a chip, e.g., a biochip. In some embodiments, the nucleic acid probes comprise a detectable label. In some embodiments, the detectable label comprises a fluorescent label.

As described herein, the therapeutically effective amount means an amount necessary to provide the indicated therapeutic benefit. As used herein, an effective amount is the amount required to confer a therapeutic effect on the treated patient. Typically, the effective amount is determined based on physical parameters such as age, surface area, weight, height, and condition of the patient. For example, a therapeutically effective amount may be from 0.01 mg to 10 g administered once (q.d.) or twice (b.i.d.) daily. In certain embodiments, the therapeutically effective amount may be administered less than once daily (e.g., every other day, weekly, etc.). In one embodiment, an effective amount is an amount that reduces activation of a RAS/RAF/MAPK pathway or component thereof within, for example, hours (e.g., 6, 12, 24), days (2, 3, 5, 6 days), weeks (e.g., 1, 2, 3, 4, 5, or 6 weeks), or months (e.g., 1, 2, 3, 4, 5, or 6 months) of administration.

The therapeutic agent can be delivered with a pharmaceutically acceptable carrier, which includes any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. The use of such media and agents for pharmaceutically active substances is well known in the art. Except insofar as any conventional media or agent is incompatible with the active ingredient, its use in the therapeutic compositions as disclosed herein is contemplated. Non-limiting examples of pharmaceutically acceptable carriers and excipients include sugars such as lactose, glucose and sucrose; starches such as corn starch and potato starch; cellulose and its analogs such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers.

### Hardware and Software

The present invention also relates to a computer system involved in carrying out the methods of the invention relating to both computations and sequencing.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. One can record results of calculations (e.g., sequence analysis or a listing of hybrid capture probe sequences) made by a computer on tangible medium, for example, in computer-readable format such as a memory drive or disk, as an output displayed on a computer monitor or other monitor, or simply printed on paper. The results can be reported on a computer screen. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

### Kits

The invention provides kits for characterizing a mutation in a target gene present in a somatic cell. For example, a kit may include primers and probes that hybridize to a target gene (e.g., PTPN11, KRAS, SOS1, BRAF, or a gene encoding a component of the Ras/Raf/MAP kinase pathway), and that may be used to characterize somatic single-nucleotide variants (SNVs), and/or measuring somatic mutation burden in a biological sample of a subject. In particular embodiments, kits include one or more reagents for single cell isolation, whole genome amplification (e.g., primers), and/or whole genome sequencing. In some embodiments, the kit includes primers for amplifying portions of proliferation-related genes previously identified as having one or more somatic mutations, and or probes that hybridize to the amplified genes.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

Recently, post-zygotic (i.e., somatic) variants have emerged as a major cause of focal epilepsies associated with focal cortical dysplasia (FCD). Pathogenic somatic variants in FCD are present in only a fraction of cells (1-10% typically), creating a mosaic with admixed mutant (variant-positive) and non-mutant cells. The level of mosaicism, as defined by variant allele frequency, in focal epilepsy usually correlates with the size and brain regional distribution of the lesion. Most somatic variants identified in FCD are seen in cases with type 2B histopathological classification and result in activation of PI3K/Akt/mTOR pathway genes that typically produce a lesion visible on MRI. However, somatic variants in non-PI3K/Akt/mTOR genes can also cause focal epilepsies, without always producing a radiographically evident dysplasia, even at high variant allele frequencies.

The hypothesis that somatic variants enriched in the hippocampus are an important pathogenic mechanism underlying drug-resistant Mesial Temporal Lobe Epilepsy (MTLE) was tested. Deep whole exome sequencing (WES) and gene panel sequencing (GPS) was utilized to identify somatic variants in the hippocampal tissue from neurosurgically-treated MTLE patients and neurotypical controls. The present data shows the presence of pathogenic somatic variants in MTLE, and shows that these variants activate the Ras/Raf/MAPK pathway—a clear distinction from the PI3K/Akt/mTOR pathway variants involved in extratemporal focal epilepsy. Additionally, significant enrichment of these pathogenic variants was identified in the affected hippocampus compared to the adjoining temporal neocortex, indicating that the frequent success of mesial temporal resection or ablation in MTLE reflects the removal of tissue that is enriched for mutant cells. These results define a new genetic pathway underlying focal epilepsy and highlight the importance of regional differences in cortical structure and development for models of epilepsy pathogenesis.

Surgical samples were obtained from 105 patients who underwent anterior medial temporal lobectomy for medication-refractory MTLE from 1988 through 2019. Clinical histopathologic findings included MTS-only (n=91), MTS+(n=6; including LEATs [n=4] or FCD [n=2] in addition to MTS), non-MTS (n=8). All patients had hippocampal tissue available. A subset also had paired temporal neocortical tissue (n=89) and blood (n=27) collected. Only fresh-frozen specimens were employed for WES or GPS and validation.

### Example 2: Pathogenic Somatic Variant Discovery and Brain Regional Enrichment Analysis

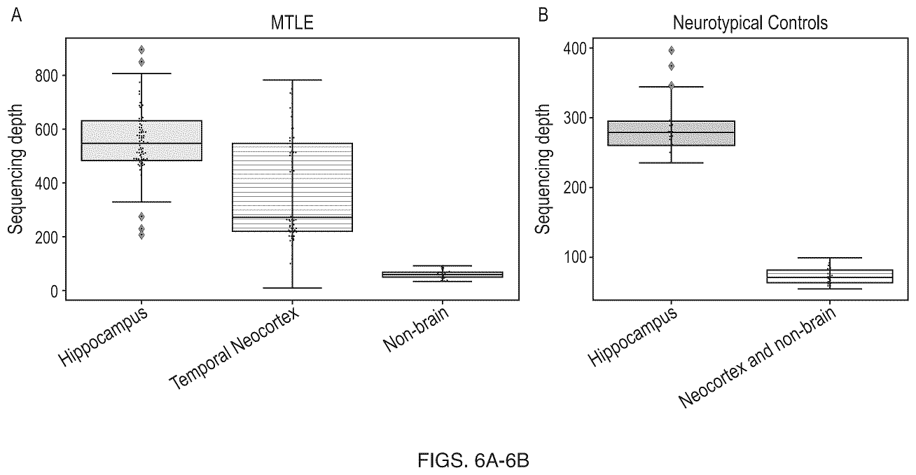

Hippocampus-derived DNA from patients underwent>500×WES or GPS (FIG. 6), followed by somatic variant calling. After careful bioinformatic measures to improve the accuracy of the somatic variant call set, all the variants previously reported as pathogenic or likely pathogenic in ClinVar were experimentally tested with amplicon sequencing and/or ddPCR. Of 21 candidate variants tested, 11 were validated. All validated pathogenic or likely pathogenic somatic variants detected in MTLE samples, and their corresponding Variant Allele Frequency (VAFs), are shown in Table 1.

TABLE 1
Pathogenic Somatic Variants in MTLE

| | | Age at Surgery | | | | | | Sequencing/ | Validation |
|---|---|---|---|---|---|---|---|---|---|
| | | (provided as | | | | | | Validation | VAF (%) |
| Participant | | a range per | | Clinical | | | Protein | VAF (%) | Temporal |
| ID | Sex | MedRxiv policy) | | Diagnosis | Gene | Variant Coordinates | Change | Hippocampus | Neocortex |
| EP17401 | M | 36-40 | years | MTS | PTPN11 | NM_002834.5: C.1507G > A | p.G503R | 2.4/1.4-1.5 | <0.2 |
| EP15001 | M | 31-35 | years | MTS, LEAT | PTPN11 | NM_002834.4: c.1508G > T | p.G503V | 2.9/2.1-2.5 | <0.2 |
| EP22101 | M | 21-25 | years | MTS | PTPN11 | NM_002834.5: c.417G > C | p.E139N | 3.3/3.3 | 0.8-0.9 |
| EP14801 | M | 16-20 | years | MTS | NF1 | NM_001042492.3: c.654 + 1G > A | Altered | 2.2/2.0 | 0.9 |
| | | | | | | NM_001042492.3: c.499_502del | splicing | germline | germline |
| | | | | | | | p.C167fs | | |
| FC8401 | F | 26-30 | years | MTS, LEAT | NF1 | NM_001042492.3: c.6852-6855del | p.Y2285fs | 6.3/6.3 | NA |
| | | | | | | NM_001042492.3: c.6904C > T | p.Q2302* | germline | germline |
| EP17501 | M | 46-50 | years | MTS | KRAS | NM_004985.5: c.35G > A | p.G12D | 1.2/0.9-0.9 | <0.2 |
| mcdbose325 | M | 0-5 | years | MTS, FCD1A | KRAS | NM_004985.5: c.35G > T^{†} | p.G12V | 19.6/18 | NA |
| EP11501 | F | 46-50 | years | MTS | SOS1 | NM_005633.4: c.810-813del | p.M269del | 1.2/0.52-0.88 | 0.6 |
| Fcde197br | F | 0-5 | years | MTS, LEAT | BRAF | NM_00433.6: c.1799T > A | p.V600E | 7.9/7.82-7.9 | NA |
| Mcdboes315 | M | 0-5 | yars | MTS, FCD2A | BRAF | NM_004333.6: c.1797A > G | p.K601E | 31.3/17.8 | 8.8 |
| EP15801 | F | 51-55 | years | MTS | SF3B1 | NM_012433.3: c.2098A > G | p.K700E | 1.7/1.5-1.7 | <0.2 |

^{†}has previously been reported in a separate cohort consisting of malformations of cortical development²³.
NA indicates no tissue was available for testing.
Validation VAFs are reported as a range (min-max) when multiple amplicons were sequenced.

Among the tissue samples with pathogenic somatic variants, 5 cases had MTS+ pathology with 2.5-23.6% mean Variant Allele Frequencies. The remaining 6 cases with MTS-only pathology had significantly lower mean Variant Allele Frequencies, ranging from 0.8 to 3.3% (p<0.01). Pathogenic somatic variants were not identified in the small non-MTS group.

Since low Variant Allele Frequency somatic variants may have been acquired at later developmental stages and be restricted to a small brain region, the presence of pathogenic somatic variants was also investigated in the unaffected temporal neocortex, when paired tissue was available. For all the variants that could be tested in both brain regions (n=8), the pathogenic variants were undetectable (<0.2% VAF, 4 patients) or less common (4 patients) in the temporal neocortex (Table 1), suggesting that variants were selectively enriched in affected hippocampal tissue (MED [IQR], 1.92 [1.5-2.7] vs 0.3 [0-0.9], p<0.05).

Notably, the PTPN11 (c.1507G>A and c.1508G>T), KRAS (c.35G>A and c.35G>T) and BRAF (c.1797A>G and c.1799T>A) variants are all located in mutational hotspots for cancer and neurodevelopmental disorders. However, except for KRAS c.35G>T and BRAF c.1799T>A which have been reported in FCDs22,23 and LEATs24, none of the other somatic variants have been previously described in focal epilepsies. Both patients with NF1 somatic variants carried a diagnosis of neurofibromatosis type 1 and had known germline variants in the NF1 gene (c.499_502del and c.6904C>T) based on clinical testing. The findings of co-existent germline and somatic variants in MTLE patients were consistent with the established double-hit mechanism in NF1-associated pathology.

To test whether pathogenic somatic variants exist in the hippocampus of neurotypical individuals, WES was performed on a cohort of 30 control hippocampal samples (FIG. 6). Since normal hippocampal tissue is rarely surgically resected, postmortem hippocampal dissections were used from individuals with no reported neurologic disease. In contrast to the MTLE cohort, no pathogenic or likely pathogenic variants were identified in the control samples.

### Example 3: Clinical Findings in Patients with Activating Ras/Raf/MAPK Somatic Variants

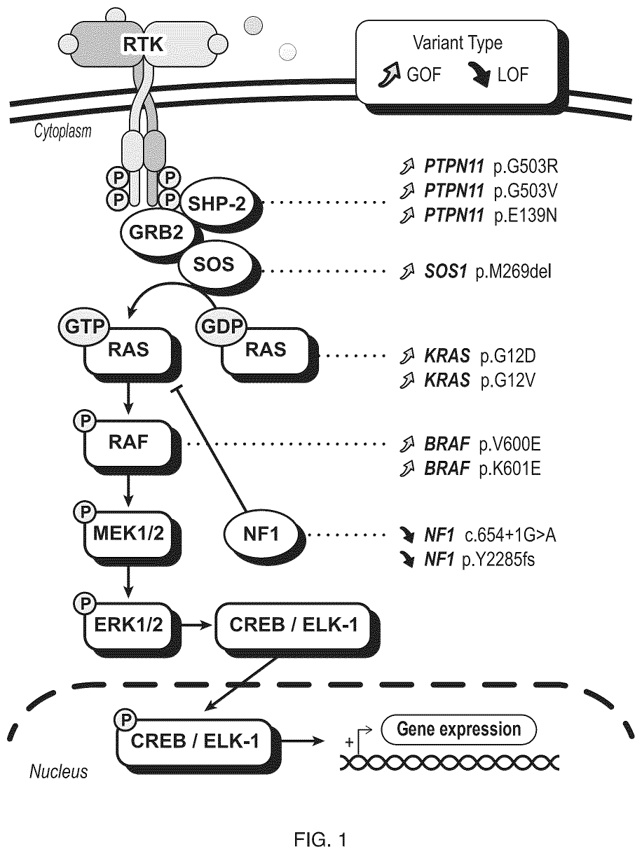

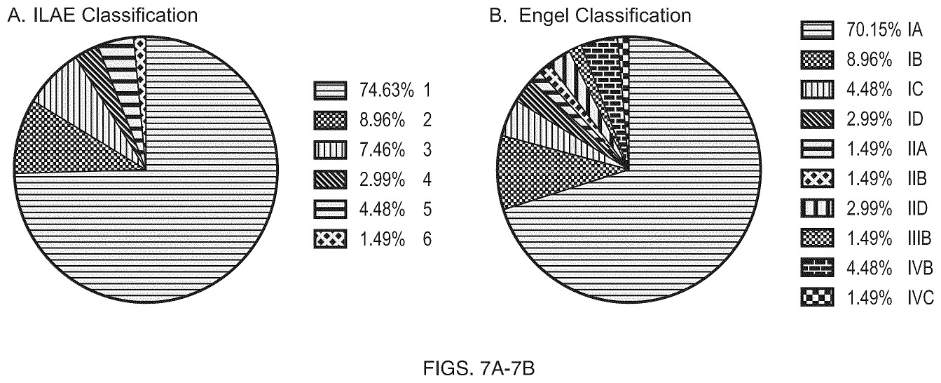

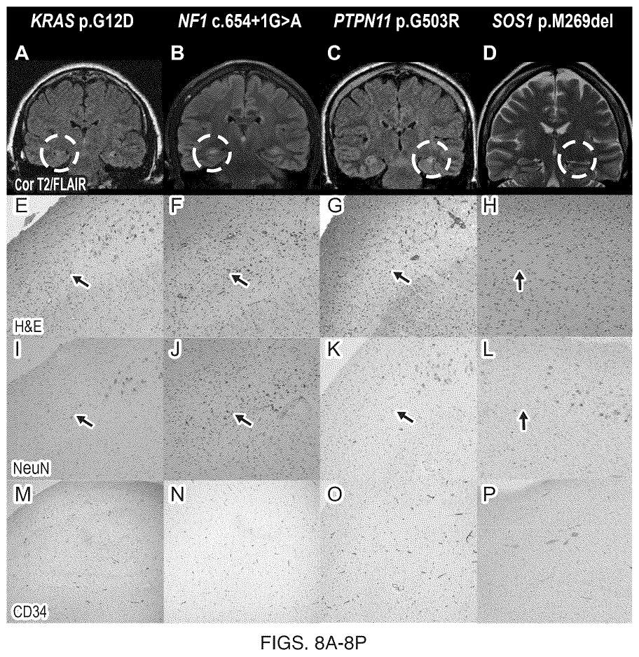

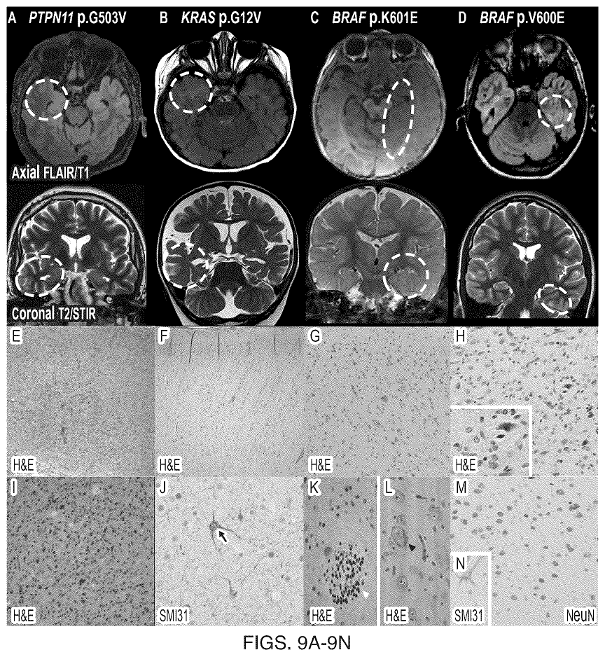

All but one variant (SF3B1 p.K700E) in Table 1 are present in Ras/Raf/MAPK pathway genes (FIG. 1A). Notably, all the variants are predicted to increase Ras/Raf/MAPK signaling via gain-of-function of a pathway activator (PTPN11, KRAS, SOS1, BRAF) or loss-of-function of a pathway repressor (NF1). To determine whether patients with activating Ras/Raf/MAPK variants have unique clinical characteristics, all available clinical data, MRI images, and histopathology slides was independently reviewed for this subgroup. All variant-positive patients were seizure-free>2 years after surgery (Engel class IA/ILAE class 1), with significantly increased likelihood of Engel class IA outcome (p<0.05) compared to the rest of the cohort (FIG. 7). All the patients with pathogenic Ras/Raf/MAPK variants had evidence of MTS; in 5 patients that was the only major imaging and histopathologic finding (FIGS. 8A-8P) while the remaining half exhibited additional findings consistent with FCDs or LEATs (FIG. 9). To further rule out the possibility of radiographically and histopathologically undetected LEATs, additional CD34 staining—a diagnostic marker for LEATs24—was performed in 4 samples with MTS-only pathology for which tissue was available. Higher than background-level CD34 staining was not observed (FIGS. 8M-8P), further confirming that Ras/Raf/MAPK variants can be present at low Variant Allele Frequencies in the affected temporal lobe in the absence of a glioneuronal tumor detected radiographically or histopathologically.

### Example 4: Enrichment of Activating Ras/Raf/MAPK Pathway Variants in MTLE

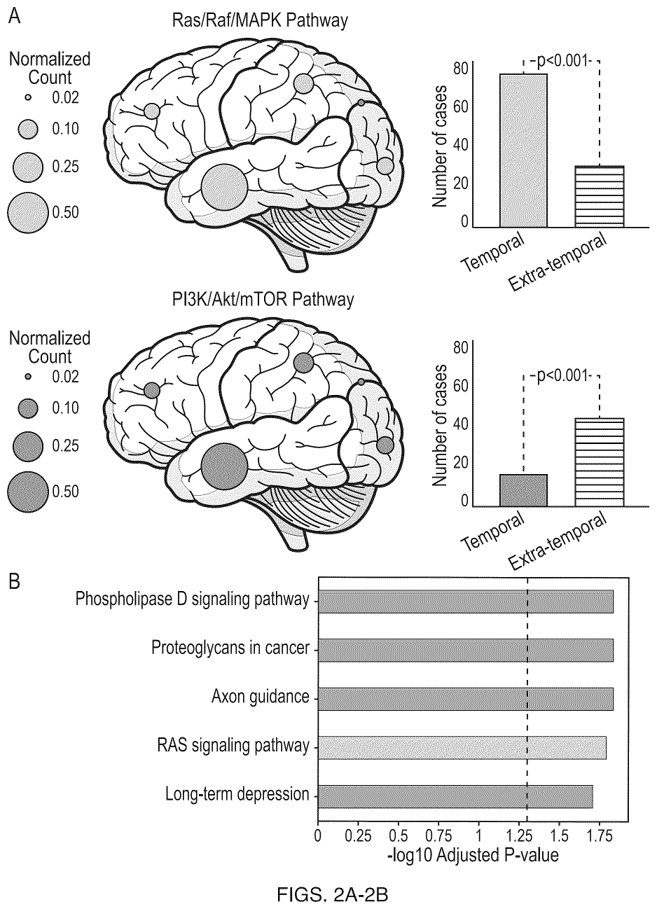

While the contribution of somatic variants to MTLE has not been previously described, most FCD-associated somatic variants reported to date are in PI3K/Akt/mTOR pathway genes whereas LEAT-associated variants often involve the Ras/Raf/MAPK pathway. To further investigate the relationship between somatic genotype and brain regional specificity, a focused retrospective review of the FCD and LEAT literature was performed. The analysis demonstrated a significant predilection of somatic Ras/Raf/MAPK variants for the temporal lobe, while PI3K/Akt/mTOR variants showed an extra-temporal predominance (FIG. 2A, p<0.001). Given that the hippocampus is the primary affected region in MTLE, this indicates that somatic variants in the Ras/Raf/MAPK pathway arising specifically in the temporal lobe confer risk for focal epilepsy.

Since the initial detection of pathogenic somatic variants relied on prior reporting of a specific variant in ClinVar, pathway enrichment analysis was next performed on all the filtered variants from the call set that were predicted to be pathogenic. Consistent with the initial observations, for somatic variants in the MTLE cohort the Ras/Raf/MAPK signaling pathway was amongst the most highly enriched pathways (FIG. 2B, adjusted p<0.05). Enrichment in this pathway was not seen in the control samples. Furthermore, it was shown that pathogenicity-enriched variants in another curated Ras/Raf/MAPK gene set (Table 2) were over-represented in the MTLE cohort (p<0.001) and not the control samples (p=1). Notably, no over-representation of PI3K/Akt/mTOR genes was seen (Table 2) in the MTLE cohort (p=1), further supporting the notion that focal epilepsies in the temporal lobe share a genetic etiology distinct from focal extra-temporal epilepsies.

TABLE 2
Curated Gene Sets for Over-representation Analysis

| | |
|---|---|
| Ras/Raf/MAPK Genes | PI3 K/A/kt/MTOR Genes |

| | | | |
|---|---|---|---|
| GRB2 | RIN1 | PIK3CA | PLD1 |
| HRAS | RIT1 | AKT3 | PLD2 |
| KRAS | SHOC2 | TSC1 | PDPK1 |
| NRAS | CBL | TSC2 | PRKCA |
| NF1 | DAB2IP | RHEB | PRR5 |
| RRAS | PRKCA | MTOR | PXN |
| RRAS2 | PRKCB | DEPDC5 | RICTOR |
| SOS1 | PRKCE | AKT1 | RPS6KA1 |
| SOS2 | PRKCZ | PTEN | RPS6KB1 |
| PTPN11 | LZTR1 | NPRL2 | RPTOR |
| BRAF | A2ML1 | NPRL3 | SGK1 |
| RAF1 | SPRY2 | AKT1S1 | SREBF1 |
| MAPK1 | SPRY3 | ATG13 | SSPO |
| MAPK3 | RALA | CCNE1 | ULK1 |
| MAP2K1 | NF2 | CDK2 | ULK2 |
| MAP2K2 | CAMK2B | CUP1 | YWHAB |
| SYNGAP1 | FGFR1 | EIF4A1 | YWHAE |
| RAS1 | FGFR2 | EIF4B | YWHAG |
| RASA2 | FGFR3 | EIF4E | YWHAH |
| RASA3 | | EIF43BP1 | YWHAQ |
| RASA4 | | DEPTOR | YWHAZ |
| RASAL1 | | DDIT4 | YY1 |
| RASGRF1 | | FBXW11 | |
| RASGRF2 | | MLST8 | |

### Example 5: Mechanism of Ras/Raf/MAPK Overactivation in MTLE-Associated PTPN11 Somatic Variants

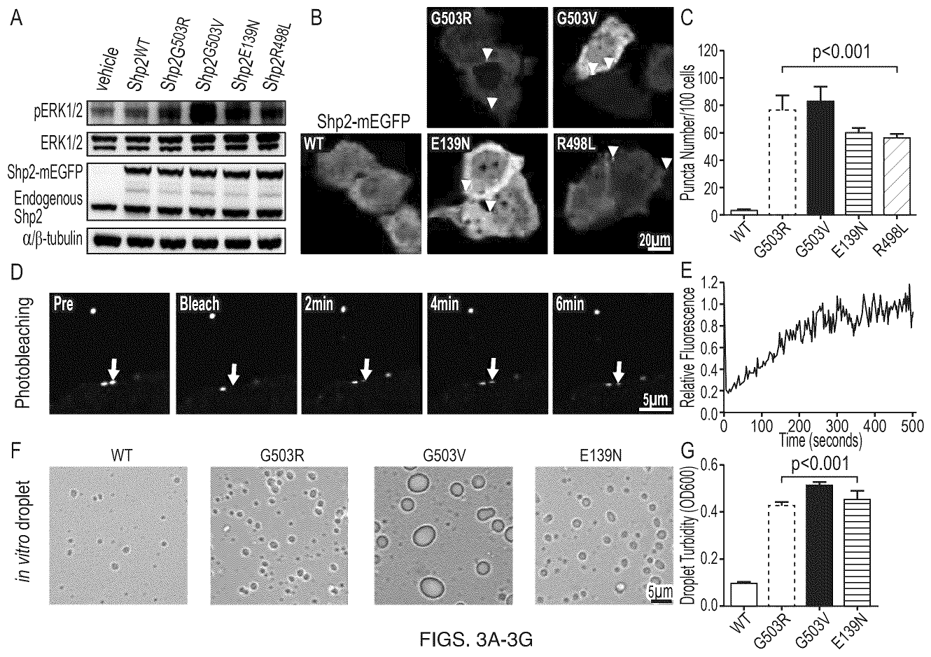

Germline PTPN11 variants, which are known causes of Noonan syndrome and related disorders, appear to enhance protein tyrosine phosphatase enzymatic activity through an acquired capability of liquid-liquid phase separation (LLPS) of the mutant Shp2 protein encoded by the PTPN11 variants. Since this gene has not been previously associated with focal epilepsy, the functional impact of MTLE-associated PTPN11 variants was evaluated by transiently expressing the human wild type (Shp2 wt) and mutant (Shp2mut) constructs in HEK293T cells and then performing immunoblotting to assess the degree of Erk1/2 (a downstream effector of the Ras/Raf/MAPK pathway) phosphorylation. Compared to cells expressing the Shp2 wt protein, increased phosphorylated Erk1/2 (pErk1/2) was detected in cells expressing Shp2mut proteins (FIG. 3A), indicating increased phosphatase activity of all the Shp2 variants consistent with constitutive activation.

To determine the mechanism through which MTLE-associated PTPN11 variants enhance phosphatase activity, whether these variants also undergo LLPS was tested. mEGFP-labeled Shp2 proteins were transiently expressed (WT, G503R, G503V, E139N, R498L) in KYSE520 cells. Remarkably, the mutant Shp2 variants formed discrete puncta in cells, whereas the wild-type protein was dispersed throughout the cell (FIG. 3B-C). Fluorescence recovery after photobleaching (FRAP) experiments showed that Shp2G503R-mEGFP puncta recovered within minutes upon photobleaching (FIGS. 3D-E), indicating that the mutant Shp2 proteins formed condensates that exhibited dynamic liquid-like behavior. To explore whether mutant Shp2 proteins could also undergo LLPS in vitro, recombinant Shp2 mutant and wild-type proteins were expressed and purified. The in vitro droplet formation assay showed that the mutant Shp2 proteins formed more and larger droplets compared to the wild-type protein in the presence of 10% (w/v) PEG3350, which was quantified using solution turbidity of OD600 (FIGS. 3F-G). These findings indicated that the MTLE-associated mutant Shp2 proteins undergo LLPS in cells and in vitro through a dominant gain-of-function mechanism that activates downstream Ras/Raf/MAPK signaling.

### Example 6: Ras/Raf/MAPK Overactivation in Affected Human MTLE Surgical Tissue

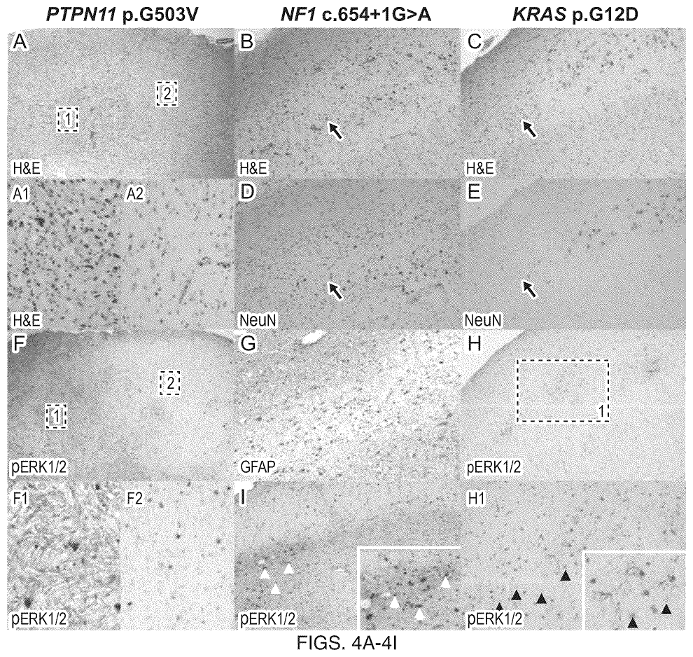

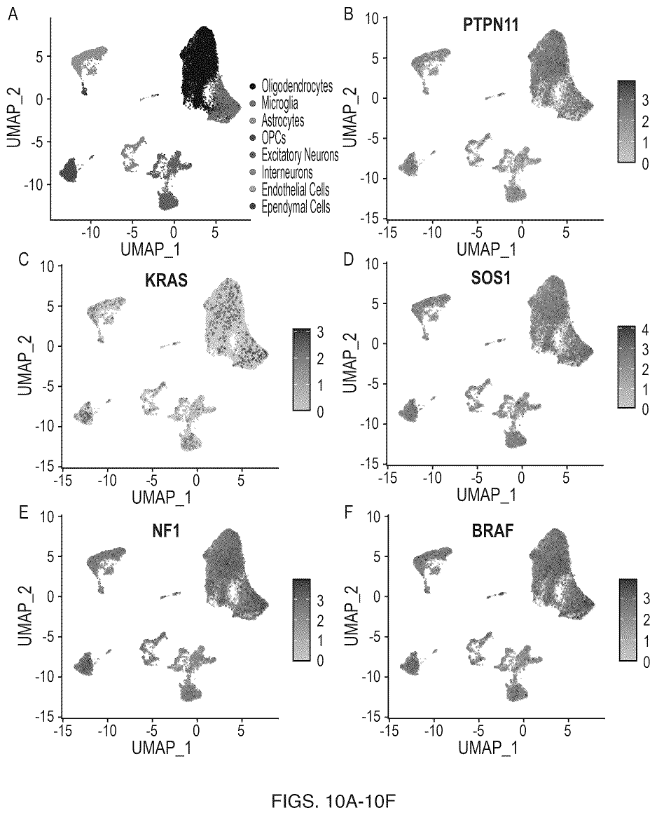

Given the enrichment of activating Ras/Raf/MAPK somatic variants in MTLE, the variant-positive cells likely play a key and potentially causal role in hippocampal epileptogenesis. In the absence of allele-specific antibodies for most of the variants found in the cohort, immunohistochemical staining for pErk1/2 (a proxy for Ras/Raf/MAPK pathway overactivation27) was used to determine the identity and localization of variant-positive cells. In brain tissue from the LEAT case associated with PTPN11 p.G503V, substantial correlation was observed between tumor density (FIG. 4A) and the degree of pErk1/2 staining (FIG. 4F), supporting the validity of this strategy in localizing variant-positive cells. Using this approach in two cases with NF1 c.654+1G>A and KRAS p.G12D variants and MTS-only pathology, it was observed that hippocampal subregions with the greatest neuronal loss showed the highest density of pErk1/2 staining (FIGS. 4B-E, H-I). Most cells with intense pErk1/2 staining in areas of neuronal loss showed glial morphology (FIGS. 4G, H1, I), which could not be explained by the differences in cell type-specific gene expression in the adult human hippocampus (FIG. 10).

Pathogenic somatic variants enriched in the hippocampus were identified, where seizures in MTLE typically originate, in 11 patients with drug-resistant MTLE, with none found in the neurotypical controls. Strikingly, all but one of these variants are predicted to activate Ras/Raf/MAPK signaling, providing strong evidence that they contribute to MTLE risk, analogous to the role of somatic PI3K/Akt/mTOR variants in FCD. The finding that even MTLE cases without evidence of dysplasia or neoplasia carry pathogenic somatic variants in the Ras/Raf/MAPK pathway significantly alters the understanding of, and the therapeutic options for, this most common indication for epilepsy surgery.

The regional specificity of epilepsy-associated Ras/Raf/MAPK pathway variants in the temporal lobe, versus PI3K/Akt/mTOR pathway variants in the extra-temporal cortex, recalls cancers where common driver mutations are characteristic to particular cell and tissue types. Hence the regional genetic specificity of focal epilepsies may represent the major differences in cellular architecture and proliferative properties between different brain regions. The higher VAFs of Ras/Raf/MAPK variants in the hippocampus compared to the lateral temporal neocortex parallels the pattern of ongoing neurogliogenesis, which continues after birth only in the dentate gyrus, and may contribute to this enrichment. Febrile seizures and head trauma, well-established risk factors for MTLE, stimulate this dentate gyrus proliferation in animal models. A similar process in humans could create a proliferative or survival advantage for cells harboring activating Ras/Raf/MAPK pathway variants, as has been described in cancer, and could provide a potential mechanism for how these risk factors may contribute to MTLE risk.

A significant association was also observed between the lower VAF of pathogenic variants and the absence of dysplasia or neoplasia on imaging and histopathology. This finding implies that patients with MTS-only pathology may have acquired their variants at later developmental stages, and therefore have fewer variant-positive cells. Patients with MTS+ pathology, in contrast, may have acquired their variants earlier resulting in higher fractions of variant-positive cells and diagnosis of drug-resistant epilepsy at a younger age, as illustrated by mcdbose325 and mcdbose315 who underwent epilepsy surgery as infants. In addition to VAF, other factors may contribute to the histopathology associated with Ras/Ras/MAPK variants. For example, EP17401 and EP15001 both have PTPN11 variants with similar VAFs that change the same amino acid, but a greater degree of Ras-MAPK activation by PTPN11 p.G503V may account for MTS+ pathology as opposed to MTS-only with p.G503R. The cellular lineage of variant-positive cells is likely an independent predictor of pathology as well. Consequently, somatic Ras/Raf/MAPK pathway variants may give rise to a spectrum of temporal lobe lesions depending on the developmental timepoint at which they were acquired, their molecular mechanisms, and the cell types of variant-positive cells, all associated with drug-resistant MTLE.

The functional data support the role of Ras/Raf/MAPK overactivation in epileptogenesis. All variant-positive patients exhibited histopathologic evidence of MTS.

This study highlights the importance of incorporating molecular testing into the MTLE diagnostic algorithm. Although it is not known yet whether MTLE-associated somatic variants can be detected less invasively, it is likely that presurgical evaluation of cell-free DNA in CSF or genomic DNA in SEEG electrodes, could guide further management steps, such as surgical approaches versus genotype-driven therapeutics. For example, in patients with left MTLE who may be at risk of significant verbal memory or language decline with an extensive resection, or in patients who do not receive surgery due to patient/provider perception or limited resources, a genotype-driven pharmacological approach may provide an additional treatment option. Since many targeted treatments for Ras/Raf/MAPK activation are already in various stages of clinical testing in cancers, these findings offer the potential to leverage some of these agents to develop the first generation of targeted therapies for molecularly characterized MTLE.

### Example 7: Testing Somatic Mutation Model by Large-Scale Sequencing with Higher Sensitivity

Large-scale sequencing with higher sensitivity was used to test the somatic mutation model. Gene-panel sequencing and drug-resistant MTLE resection was used in the case-control study to test for somatic mutations. Patients with non-lesional and lesional (mesial temporal sclerosis [MTS], focal cortical dysplasia, and low-grade epilepsy-associated tumors [LEATs: ganglioglioma, dysembryoplastic neuroepithelial tumor, and multinodular and vacuolating neuronal tumor]) drug-resistant MTLE who underwent anterior mesial temporal lobe resection were included. All non-LEAT primary brain tumors were excluded. Mean coverage for the large-scale sequencing conducted here was >1000×. The cohort included both subjects found to have MTLE (n=558) and neurotypical controls (n=74). Somatic mutations found in the MTLE subjects are shown in Table 3, below.

TABLE 3
Table of Somatic Mutations Found in MTLE Subjects from Large-scale Sequencing

| Ras-MAPK | | | | PI3K-mTOR | FCD | DEE | |
|---|---|---|---|---|---|---|---|
| GRB2 | RAF1 | RASAL1 | PRKCZ | PIK3CA | SLC3542 | PCDH19 | CASK |
| HRAS | MAPK1 | RASGRF1 | LZTR1 | AKT3 | | SCN1A | CDKL5 |
| KRAS | MAPK3 | RASGRF2 | A2ML1 | TSC1 | | SCN1B | MECP2 |
| NRAS | MAP2K1 | RIN1 | SPRY2 | TSC2 | | SCN2A | STXBP1 |
| NF1 | MAP2K2 | RIT1 | SPRY3 | RHEB | | SCN8A | LGI1 |
| RRAS | SYNGAP1 | SHOC2 | FGFR1 | MTOR | | GABRA1 | |
| RRAS2 | RASA1 | CBL | FGFR2 | DEPDC5 | | GABBR2 | |
| SOS1 | RASA2 | DAB2IP | FGFR3 | AKT1 | | GABRB2 | |
| SOS2 | RASA3 | PRKCA | RALA | PTEN | | KCNQ2 | |
| PTPN11 | RASA4 | PRKCB | NF2 | NPRL2 | | KCNQ3 | |
| BRAF | CAMK28 | PRKCE | | NPRL3 | | ARX | |

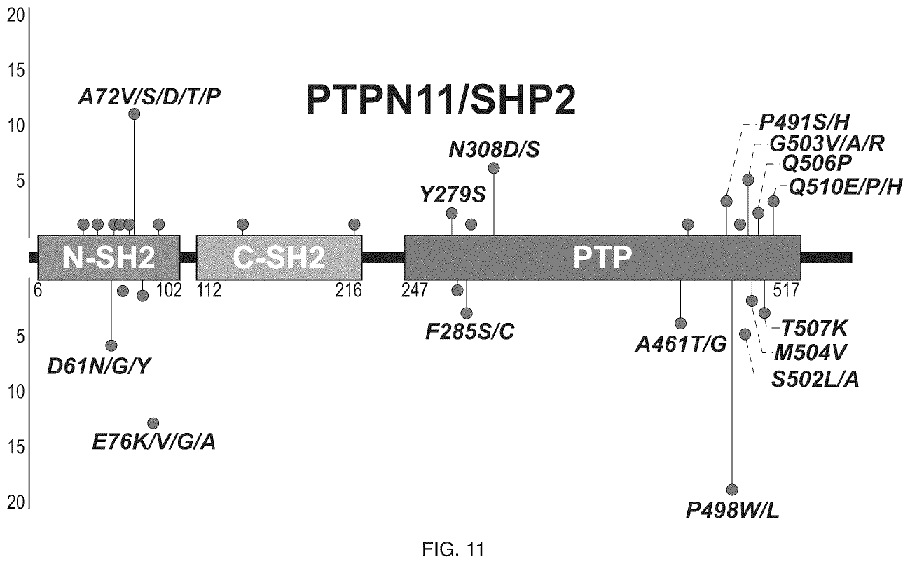

The data revealed>100 mutations in PTPN11 alone (>15% of cases). Examples of frequent, highly recurrent, mutations PTPN11/SHP2 mutations found through this sequencing are shown in FIG. 11. All of these PTPN11 mutation swere found to be missense, many were found to be recurrent, and many were known to be activating. The total yield of PTPN11/RAS/RAF/MAPK mutations (>30%) exceeded the yield of MTOR variants previous shown in FCD with panel sequencing (26%, D'Gama et al., 2017). 75% of mutation-positive cases showed MTS only, and mutation in 0.5-3% of cells.

Next, the validity of the somatic mutation model was investigated. In short, it was hypothesized that risk factors for MTLE stimulate proliferation favoring RAS-activated clones. This higher fraction of RAS-mutant cells would, in turn, increase epilepsy risk. This model would explain both why “seizures beget seizures” and why the temporal lobe shows a predilection for epilepsy.

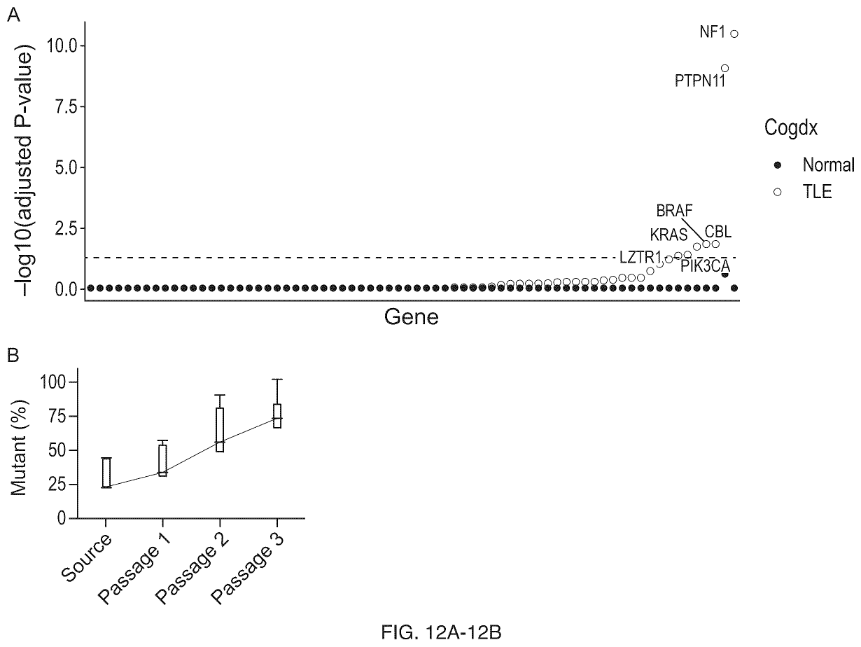

Methods previously used to distinguish cancer “driver” versus “passenger” mutations revealed that PTPN11/RAS/RAF/MAPK mutations show clonal selection in the brain of MTLE subjects (FIG. 12A). When further tested in iPSC cell lines, 50/50 mixing of isogenic wild-type and mutant (PTPN11 G503R) cell lines showed that the mutant cells had a distinct advantage (FIG. 12B).

These results indicated that the MTLE brain selects for further PTPN11/RAS/RAF/MAPK mutations.

The results described herein above, were obtained using the following methods and materials.

### Study Participants

This case-control study was designed to investigate the association between somatic genetic variants and MTLE. Patients with non-lesional and lesional (mesial temporal sclerosis [MTS], focal cortical dysplasia, and low-grade epilepsy-associated tumors [LEATs: ganglioglioma, dysembryoplastic neuroepithelial tumor, and multinodular and vacuolating neuronal tumor]) drug-resistant MTLE who underwent anterior mesial temporal lobe resection were included. All non-LEAT primary brain tumors were excluded. Neurotypical control brain tissue was obtained from the NIH NeuroBioBank at the University of Maryland, Baltimore, and the University of Miami, and the European Epilepsy Brain Bank.

### Oversight and Study Procedures

The main study group comprised MTLE patients at Yale-New Haven Hospital, Boston Children's Hospital, Austin Hospital, and the Royal Melbourne Hospital. Written informed consent was obtained from all patients or their guardians with local Institutional Review Board or Ethics Committee approval. All research performed with samples obtained from patients was approved by the Institutional Review Board at Boston Children's Hospital. Participants IDs are assigned by the research team are not identifiable personal or clinical information.

### Target Capture, Sequencing, and Somatic Variant Analysis

WES and GPS (mean depth>500×) was performed on DNA extracted from fresh-frozen brain tissue or non-brain tissue to detect somatic mutations, which were independently validated with amplicon sequencing or droplet digital Polymerase Chain Reaction (ddPCR).

### Cellular and Molecular Studies

Cell lines were transfected with wild-type and mutant human gene constructs and underwent immunoblotting and liquid-liquid phase separation assays. Additional histopathology experiments were performed on archival paraffin-embedded tissue as needed.

### Statistical Analysis

Pairwise comparisons were made with Fisher's exact test, Student's t-test, Wilcoxon rank-sum and signed-rank tests, and the binomial test; p-values less than 0.05 were considered statistically significant. Pathway enrichment analysis17 and gene set over-representation analysis were evaluated using the hypergeometric test; False Discovery Rate (FDR)-adjusted p-values less than 0.05 were considered statistically significant.

### Confirmation of Clinical, Radiologic, and Histopathologic Findings

Clinical and surgical information was obtained from patients' charts by the authorized study team. All diagnoses were confirmed by the study pathologists, radiologists, and neurosurgeons. Radiology images and histopathology slides for patients with tissue harboring pathogenic somatic variants were additionally reviewed by an independent expert neuroradiologist (EY) and neuropathologist (IB), respectively.

### Brain Tissue Procurement

Brain tissue was labeled as hippocampus or temporal neocortex in the OR and then evaluated and allocated for research by the clinical neuropathology team. While in all cases anterior medial temporal tissue and anterior temporal neocortical tissue were collected, there may have been some variability in the exact location that was sampled for genomic DNA extraction.

### Genomic DNA Extraction

Genomic DNA from fresh-frozen surgical samples and whole-blood was extracted using Chemagic DNA Tissue 100 H24 kit (Perkin Elmer), DNeasy Blood & Tissue kits (Qiagen), All Prep DNA/RNA kit (Qiagen), QIAamp DNA Blood kits (Qiagen), or a standard phenol chloroform extraction (Green M R, Sambrook J. Isolation of High-Molecular-Weight DNA Using Organic Solvents. Cold Spring Harb Protoc. Cold Spring Harbor Laboratory Press; 2017 Apr. 1; 2017(4)). Standard quality and purity assessments were conducted via determination of the 260/280 nm ratios for values of 1.7-2.0. All the genomic DNA samples were quantified using Qubit or PicoGreen (ThermoFisher Scientific) for proper assessment of double stranded DNA concentration.

### Whole-Exome Sequencing

Genomic DNA derived from brain and non-brain tissue underwent whole-exome capture using IDT xGen Exome Research Panel v1+custom spike-in probes (IDTERPvlcustom), IDT xGen Exome Research Panel v1+mitochondrial genome spike-in probes (IDTERPv1mtDNA), or Twist Human Comprehensive Exome v1 (TwistHCEv1). For the samples prepared using the Twist capture probes, IDT xGen UDI-UMI adapters were substituted in to improve variant calling accuracy. The prepared paired-end whole-exome libraries were sequenced on NovaSeq 6000 at Yale Center for Genome Analysis, Harvard Biopolymers Facility, or Columbia University Irving Medical Center.

### Gene-Panel Sequencing

Genomic DNA derived from brain tissue underwent gene-panel sequencing. Starting from recurrent cancer hotspot mutations in a commercially-available panel (Cancer Hotspot v2, Thermo Fisher) and cancer-linked mutations in MTOR-pathway genes (COSMIC, accessed November 2018), gain-of-function SNPs were selected, prioritizing variants occurring multiple times in COSMIC datasets and submitting top variants for Ampliseq pool design with manufacturer-designed software (Ampliseq, Thermo Fisher). To generate Ampliseq libraries, 10 ng of genomic DNA was added to 1× final Phusion U Hot Start Master Mix (Thermo Fisher), 0.9× Ampliseq primer pool, and 80 ng ET SSB (NEB), undergoing 17 cycles of PCR before standard Illumina library preparation. The panel was sequenced on a Nextseq 500 high output flow cell at Harvard Biopolymers Facility.

### Alignment and Preprocessing of Sequencing Data

### IDT Whole-Exome Sequencing

All the whole-exome sequencing data from cases and controls were aligned to hg38 reference genome (GRCh38.p13) using Burrows-Wheeler Aligner (BWA, version 0.7.17) (Li H, Durbin R. Fast and accurate short read alignment with Burrows-Wheeler transform. Bioinformatics. 2009 Jul. 15; 25(14):1754-1760). The GATK Best Practices Workflow (Van der Auwera, GA OB. Genomics in the Cloud: Using Docker, GATK, and WDL in Terra. 1^{st} Edition. O'Reilly Media; 2020) was used for the remainder of preprocessing steps. Briefly, duplicates were marked using Picard (Picard Tools—By Broad Institute [Internet]. [cited 2022 May 11]. Available from: http://broadinstitute.github.io/picard/) (version 2.18.14) MarkDuplicates. The bam tags were then sorted and fixed and base quality recalibration was performed using GATK (Van der Auwera, GA OB. Genomics in the Cloud: Using Docker, GATK, and WDL in Terra. 1^{st} Edition. O'Reilly Media; 2020) (version 4.1.9.0) SortSam, SetNmMdAndUqTags, BaseRecalibrator, and ApplyBQSR.

### Twist Whole-Exome Sequencing

Following the best practices for data analysis when using UMI adapters to improve variant detection (Best practices for data analysis when using UMI adapters to improve variant detection [Internet]. Integrated DNA Technologies. [cited 2022 May 11]. Available from: https://www.idtdna.com/pages/education/videos/detail/best-practices-for-data-analysis-when-using-umi-adapters-to-improve-variant-detection), the resulting binary base call (bcl) file was demultiplexed for each sample and converted to an unaligned bam file with UMI information using Picard (version 2.18.14) ExtractIlluminaBarcodes and IlluminaBasecallsToSam functions. The reads were then aligned from the unaligned bam file to the hg38 reference genome (GRCh38.p13) and included the UMI tags from unmapped reads using Picard SamToFastq and MergeBamAlignment, BWA (version 0.7.17), and Samtools (Li H, Handsaker B, Wysoker A, et al. The Sequence Alignment/Map format and SAMtools. Bioinformatics. 2009 Aug. 15; 25(16):2078-2079; Li H. A statistical framework for SNP calling, mutation discovery, association mapping and population genetical parameter estimation from sequencing data. Bioinformatics. 2011 Nov. 1; 27(21):2987-2993) (version 1.19) merge functions. The reads were then grouped by UMIs with the fgbio (Smith TS, Heger A, Sudbery I. UMI-tools: Modelling sequencing errors in Unique Molecular Identifiers to improve quantification accuracy. Genome Res. 2017 Jan. 18; gr.209601.116) (version 1.4.0) GroupReadsByUmi function. The consensus reads (n=1) were then generated with fgbio CallMolecularConsensusReads function, sorted them with Picard SortSam, realigned them using BWA, and included the UMI tags from unmapped reads using Picard MergeBamAlignment. Lastly, the resulting consensus bam files were filtered with fgbio⁸ FilterConsensusReads (min-reads=1, min-base-quality=20, max-no-call-fraction=0.05, min-mean-base-quality=10). To produce high-quality variant calls, additional preprocessing steps weew next performed on the filtered consensus bam files. First, the bam tags were sorted and fixed using GATK (version 4.1.9.0) SortSam and SetNmMdAndUqTags. Second, local indel realignment was performed with GATK (version 3.8.1.0) RealignerTargetCreator and IndelRealigner. Last, base quality score recalibration was performed with GATK (version 4.1.9.0) BaseRecalibrator and ApplyBQSR.

### Custom Gene-Panel Sequencing

All the whole-exome sequencing data from cases and controls were aligned to hg38 reference genome (GRCh38.p13) using BWA (version 0.7.17). The bam tags were then sorted and fixed and base quality recalibration was performed using GATK (version 4.1.9.0) SortSam, SetNmMdAndUqTags, BaseRecalibrator, and ApplyBQSR.

### Somatic Single Nucleotide Variant (SNV) and Small Insertion-Deletion Variant (INDEL) Calling

### Unpaired Calling

Unpaired somatic variant calling was performed using GATK (version 4.1.9.0) MuTect2 (Cibulskis K, Lawrence M S, Carter S L, et al. Sensitive detection of somatic point mutations in impure and heterogeneous cancer samples. Nat Biotechnol. 2013 March; 31(3):213-219. PMCID: PMC3833702) using the hippocampal preprocessed bam files as “tumor”. The raw MuTect2 vcf files were filtered using GATK (version 4.1.9.0) FilterMutectCalls.

### Paired Calling

For all the hippocampal samples with paired non-brain or neocortical tissue, the preprocessed bam files for the paired tissue were downsampled to 50× using sambamba (Tarasov A, Vilella A J, Cuppen E, Nijman I J, Prins P. Sambamba: fast processing of NGS alignment formats. Bioinformatics. 2015 Jun. 15; 31(12):2032-2034.) (version 0.7.1), mosdepth (Pedersen B S, Quinlan A R. Mosdepth: quick coverage calculation for genomes and exomes. Bioinformatics. 2018 Mar. 1; 34(5):867-868) (version 0.3.2), and customized python scripts. The downsampling was performed in order to minimize the chance of filtering out a shared somatic variant present in both the hippocampus and a paired tissue. Then paired somatic variant calling was performed with GATK (version 4.1.9.0) MuTect2 and Strelka2 (Kim S, Scheffler K, Halpern A L, et al. Strelka2: fast and accurate calling of germline and somatic variants. Nat Methods. Nature Publishing Group; 2018 August; 15(8):591-594) (version 2.9) using the hippocampal bam files as “tumor” and the downsampled paired tissue bam files as “normal”. The raw MuTect2 vcf files were filtered using GATK (version 4.1.9.0) FilterMutectCalls. Finally, the filtered MuTect2 and Strelka2 call sets were merged to create a union call set with high sensitivity for somatic variant discovery.

Paired Calling with a Panel of Normals (PON)

For pathway enrichment analysis where accuracy was prioritized over sensitivity, a PON approach was used for variant calling to eliminate the false positive somatic calls caused by technical factors and error-prone genomic regions. Following a previously published strategy (Dou Y, Kwon M, Rodin R E, et al. Accurate detection of mosaic variants in sequencing data without matched controls. Nature Biotechnology. Nature Publishing Group; 2020 March; 38(3):314-319), for each case a unique PON was created using all the somatic variant call sets in the same cohort excluding the case that was being evaluated. This was achieved using GATK (version 4.1.9.0) GenomicDBImport followed by CreateSomaticPanelofNormals. Then paired somatic variant calling was performed using GATK (version 4.1.9.0) MuTect2 as described above, this time including a PON as an additional input. The raw MuTect2 vcf file was filtered by GATK (version 4.1.9.0) FilterMutectCalls. Since somatic variant calling could be affected by sequencing depth, for Gene Set Overrepresentation Analysis (described below) where cases and controls were directly compared side by side, the preprocessed bam files for the MTLE hippocampal tissue were downsampled to 284× to achieve the same mean sequencing depth for MTLE and control bam files.

### Somatic Variant Annotation

All the variants were annotated using snpEff (Cingolani P, Platts A, Wang L L, et al. A program for annotating and predicting the effects of single nucleotide polymorphisms, SnpEff. Fly (Austin). 2012 Apr. 1; 6(2):80-92. PMCID: PMC3679285) (version 5.1). The databases included were the latest versions of gnomAD (Karczewski K J, Francioli L C, Tiao G, et al. The mutational constraint spectrum quantified from variation in 141,456 humans. Nature. Nature Publishing Group; 2020 May; 581(7809):434-443), ExAC (Karczewski K J, Weisburd B, Thomas B, et al. The ExAC browser: displaying reference data information from over 60 000 exomes. Nucleic Acids Res. 2017 Jan. 4; 45(Database issue):D840-D845. PMCID: PMC5210650), ClinVar (Landrum M J, Lee J M, Benson M, et al. ClinVar: improving access to variant interpretations and supporting evidence. Nucleic Acids Research. 2018 Jan. 4; 46(D1):D1062-D1067), Rare Exome Variant Ensemble Learner (REVEL) (Ioannidis N M, Rothstein J H, Pejaver V, et al. REVEL: An Ensemble Method for Predicting the Pathogenicity of Rare Missense Variants. The American Journal of Human Genetics. 2016 Oct. 6; 99(4):877-885), SNVs in Evolutionary model of Variant Effect (EVE) (Frazer J, Notin P, Dias M, et al. Disease variant prediction with deep generative models of evolutionary data. Nature. Nature Publishing Group; 2021 November; 599(7883):91-95), Catalogue Of Somatic Mutations In Cancer (COSMIC, v95) (Tate J G, Bamford S, Jubb H C, et al. COSMIC: the Catalogue Of Somatic Mutations In Cancer. Nucleic Acids Research. 2019 Jan. 8; 47(D1):D941-D947), and SpliceAI (Jaganathan K, Kyriazopoulou Panagiotopoulou S, McRae J F, et al. Predicting Splicing from Primary Sequence with Deep Learning. Cell. 2019 Jan. 24; 176(3):535-548.e24) (version 1.3.1).

### Pathogenic Somatic Variant Discovery

Since establishing pathogenicity in somatic variants can be challenging and frequently requires extensive functional validation, the initial pathogenic variant discovery was limited to known variants that were previously reported in ClinVar as “pathogenic” or “likely pathogenic.” Only variants in genes with expression in the hippocampus based on The Genotype-Tissue Expression (GTEx) Project (The Genotype-Tissue Expression (GTEx) project. Nat Genet. 2013 June; 45(6):580-585. PMCID: PMC4010069) were included in further analysis. Given the enrichment of Ras/Raf/MAPK variants based on the initial analysis, the raw (unfiltered) MuTect2 call sets were also independently screened for the presence of pathogenic or likely pathogenic Ras/Raf/MAPK variants. To remove the likely false positives, candidate variants were visually inspected using Integrative Genomics Viewer (IGV) (Robinson J T, Thorvaldsdóttir H, Winckler W, et al. Integrative Genomics Viewer. Nat Biotechnol. 2011 January; 29(1):24-26. PMCID: PMC3346182) and only variants that met the following criteria were submitted for experimental confirmation:
Variant Allele Frequency (VAF)<0.3
Presence on at least 2 alignments with different start positions
Minimum 3 alternative (ALT) reads on forward and reverse orientations
Absence from error-prone genomic regions with multiple other somatic variants within a 50 bp window.
   Experimental confirmation was performed using amplicon sequencing or ddPCR as discussed separately.

### Pairwise Statistical Tests of VAFs for Pathogenic Ras/Raf/MAPK Variants

Pairwise comparisons of VAFs were conducted for pathogenic Ras/Raf/MAPK in:
Cases with MTS versus cases with MTS+ pathology (Two-sided Wilcoxon rank-sum test)
Cases with both hippocampal and temporal neocortical tissue (Two-sided Wilcoxon signed-rank test)

To determine the appropriate statistical test, for each pair of VAFs, it was first tested whether they were normally distributed using the Shapiro test. The equality of variances of the pair (homoscedasticity) were next tested. If both were normally distributed (Shapiro test p-value>=0.05), the Bartlett test was used; if at least one was not normally distributed (Shapiro test p-value<0.05), the Levene test was used. Last, the equality of mean was tested based on the previous tests of normality and homoscedasticity. If the pair passed both tests (Bartlett test p-value>=0.05), Student's t-test was used; if the pair failed either test (Shapiro test p-value<0.05 or Bartlett test p-value<0.05 or Levene test p-value<0.05), the Wilcoxon rank-sum test was used. In both scenarios, the pair was determined to be significantly different if p-value<0.05. All statistical tests were implemented using the Python SciPy library (Jones E, Oliphant T, Peterson P, Others. SciPy: Open Source Scientific Tools for Python [Internet]. 2001. Available from: http://www.scipy.org).

### Pairwise Statistical Test for Surgical Outcome

To determine whether the 2-year surgical outcome is different for the Ras/Ras/MAPK variant-positive patients compared to the rest of the cohort, cases were only included with sufficient clinical information at the 2-year postsurgical timepoint (n=67, eFigure 2). The statistical likelihood of Engel class IA outcome vs. non-Engel class IA outcome was then tested in patients with pathogenic Ras/Ras/MAPK variants compared to the rest of the cohort using Fisher's exact test.

### Pathway Overrepresentation Analysis

The initial approach for pathogenic variant discovery relied on variants that were previously reported as pathogenic or likely pathogenic, which yielded an excess of Ras/Raf/MAPK pathway variants. However, to assess the burden of all somatic SNVs with pathogenicity potential—known and unknown—a less biased and comprehensive strategy was employed as follows:

### Creating a High-Quality Call Set

To minimize the number of false positives and create an accurate set of somatic variants for downstream analyses, the paired MuTect2-PON call set was used, a sample was filtered out that had an excessive number of variant calls due to contamination, and the following filters were applied to the remaining samples:
Variant Allele Frequency (VAF)<0.3
Presence in only one individual in the cohort
Population allele frequency<0.001 in gnomAD and ExAC
Presence on at least 2 alignments with different start positions
Supported by minimum 10 ALT reads
Minimum 3 ALT reads on forward and reverse orientations
Absence from error-prone genomic regions (overlapping RepeatMasker annotations, segmental duplications as annotated in UCSC SegDup, and germline indels)
Absence of an adjacent homopolymer repeat with at least 3 units

Filters 4-8 were carried out using an in-house computational pipeline. The accuracy of the pipeline was independently tested using visual inspection of a separate call set using IGV. But to also validate the approach experimentally, a random set of variants was evaluated from the MTLE and control cohorts using amplicon sequencing. All base substitution types were included in this evaluation, but more T>A/A>T transversions were added given an unexpected excess of these variants in the control cohort. For each type of substitution, SNV samples were drawn from all the filtered SNVs that are representative of the overall VAF distribution, i.e., since VAF ranges from 0 to 0.3, the range was broken into 6 discrete bins of 0.05 and the number of randomly selected SNVs in each bin was proportional to the number of SNVs in each bin. For the MTLE cohort, 56 out of the 65 tested variants (86.2%) were experimentally validated. For the control cohort, 33 out of the 68 variants (48.5%) were experimentally validated. However, the excess T>A/A>T transversions in the control cohort, likely introduced during library prep, accounted for 60% of the unvalidated control variants, otherwise the two cohorts had similarly high experimental confirmation rates.

### Pathogenicity Enrichment

To enrich for somatic SNVs with pathogenicity potential in the high-quality call set, REVEL, EVE, COSMIC, and SpliceAI annotations were relied on. Among the databases, REVEL and SpliceAI provide a pathogenicity score between 0 and 1 for each variant in the database, respectively, and the higher the score, the more likely the variant is to be pathogenic. EVE provides three classifications: “benign”, “pathogenic”, “uncertain”. EVE class80 annotation, which was used, classifies 80% of the variants as pathogenic or benign. All the variants in COSMIC are presumed to be cancer-related and thus pathogenic. Given that the majority (>90%) of the missense variants have REVEL annotations and that only a limited number of variants have EVE and COSMIC annotations, the following criteria were used to enrich for pathogenicity:
1. REVEL score>0.7 if only annotated by REVEL
2. REVEL score 0.6-0.7 and EVE class80 classification as pathogenic or uncertain, unless present in COSMIC
3. If REVEL score unavailable and EVE class80 classification as pathogenic, unless present in COSMIC
4. SpliceAI score>0.8

### Pathway Enrichment Analysis

To evaluate for the specific biological pathways that may be affected by somatic variants in MTLE, the overrepresentation of MTLE pathogenicity-enriched somatic SNVs were tested for in gene sets curated by the Kyoto Encyclopedia of Genes and Genomes (KEGG). To perform this analysis KOBAS (Bu D, Luo H, Huo P, et al. KOBAS-i: intelligent prioritization and exploratory visualization of biological functions for gene enrichment analysis. Nucleic Acids Research. 2021 Jul. 2; 49(W1):W317-W325) was used, an online tool, and KEGG pathways were selected as the desired option. Because KOBAS does not take duplicate gene name inputs, all the genes with more than one pathogenicity-enriched somatic variant were counted only once (e.g., PTPN11). Hypergeometric test/Fisher's exact test was used for statistical analysis, and Benjamini and Hochberg (1995) was used to correct for multiple hypothesis testing in KOBAS.

### Gene Set Overrepresentation Analysis

To test specifically for over-representation of pathogenicity-enriched somatic variants in specific gets sets, two gene lists were curated: Ras/Raf/MAPK (n=43 genes) and PI3K/Akt/mTOR (n=46 genes) as shown in. These genes were selected based on a review of other available gene lists (“RAS Pathway v2.0” published on the National Cancer Institute website and “MTOR Signaling Pathway” from the Pathway Interaction Database (Schaefer CF, Anthony K, Krupa S, et al. PID: the Pathway Interaction Database. Nucleic Acids Res. 2009 January; 37(Database issue):D674-679. PMCID: PMC2686461), accessed on Apr. 1, 2022) based on their relevance to neurologic diseases and expression in the brain using the GTEx database. Then the hypergeometric test was used to assess for over-representation of pathogenicity-enriched somatic variants in these gene sets against a background of approximately 19,300 genes targeted by the whole-exome sequencing panel. The pathogenicity-enriched MTLE variants overlapped with 2 Ras/Raf/MAPK genes and 0 PI3K/Akt/mTOR genes out of 13 total genes, whereas in the control call set 0 Ras/Raf/MAPK and PI3K/Akt/mTOR genes were detected out of 34 total genes (n=34). It is important to note that some of the experimentally confirmed pathogenic Ras/Raf/MAPK variants in the MTLE cohort, were filtered out due to a combination of downsampling and the rigorous filtering that was required for creating a high-quality call set.
Shown below are the specific calculations:
For Ras/Raf/MAPK ene overrepresentation in MTLE:

$$p = 1 - {\sum }_{i = 0}^{1} \frac{( \begin{matrix}43 \\ i\end{matrix} ) ( \begin{matrix}19257 \\ 13 - i\end{matrix} )}{( \begin{matrix}19300 \\ 13\end{matrix} )} = 4.09 \times 1 {0}^{- 4}$$

For all other scenarios, the summation term does not exist because there is 0 overlap, so:

$$p = 1$$

The statistical tests were repeated with and without the excess T>A/A>T transversions in the control cohort and it was confirmed that it did not change the results of this analysis. Since no variants in Ras/Raf/MAPK or PI3K/Akt/mTOR genes were detected in the control cohort, the summation term remained at 0 and p=1.

### Experimental Confirmation of Candidate Variants

### Amplicon Validation

Custom primers were designed for each candidate variant using the default settings in Primer3¹⁻² to generate 150-300 bp amplicons. The primers were commercially synthesized (IDT) and tested on human genomic DNA (Promega) to confirm generation of only one amplicon product at the expected size. Then 10-50ng of genomic DNA from patients (based on sample availability) were used to create amplicons for sequencing, purified using 2× AMPure XP, and run on a gel for quality control. Amplicons from different samples were pooled together and Illumina sequenced to achieve at least 10,000 reads per each unique amplicon. The raw reads were aligned to the reference genome (hg38) using BWA (version 0.7.17) and the visualized on IGV to confirm the presence of each candidate variant. The variant allele frequencies were calculated based on the total number of REF and ALT alleles. Based on the limitations of PCR amplification and Illumina sequencing, the cutoff for confirmation was set at 0.3% AF.

ddPCR Validation

For ddPCR, commercially available TaqMan primer/probe mixes were ordered for BRAF V600E (C_151552107_10, Thermo Fisher) flanking the mutation site. First, 10 ng genomic DNA was digested using the HindIII restriction enzyme. Then the digested DNA, TaqMan primer/probe mix, and ddPCR Supermix for Probes (Bio-Rad) was used to perform ddPCR according to the guidelines published in Droplet Digital PCR Applications Guide (Bio-Rad). Finally, the ddPCR data was analyzed using the software QuantSoft (Bio-Rad). The variant allele frequencies were calculated based on the total number of REF and ALT alleles.

### Retrospective Review of Somatic Ras/Raf/MAPK and PI3K/Akt/MTOR Variants in Focal Epilepsy

To perform a retrospective review of published lesional focal epilepsy cases with Ras/Raf/MAPK and PI3K/Akt/mTOR somatic variants, a search on pubmed.gov (National Library of Medicine: National Center for Biotechnology Information) was performed using the following criteria:
1. Cases that were published from 2012 through 2022
2. Specific location of the lesion on the brain was identified in the paper
3. Pathogenic somatic variants were identified in Ras/Raf/MAPK and PI3K/Akt/mTOR genes
   The following search terms in pubmed were used:
1. “FCD” “somatic” “mutation” “mtor”
2. 36 search results
3. 14 papers met the above criteria
4. “mutation” “epilepsy” “fcd”
5. 45 search results
6. 3 papers met the above criteria
7. “mtor” “focal cortical dysplasia”
8. 139 search results
9. 1 paper met the above criteria
   1. “ras” “mutation” “ganglioglioma”
   2. 6 search results
   3. 2 papers met the above criteria
   4. raf mutation ganglioglioma
   5. 90 search results
   6. 9 papers met the above criteria
   7. “braf” “mutation” “dnet”
   8. 6 search results
   9. 1 paper met the above criteria
   10. mutation ganglioglioma
   11. 12 search results
   12. 3 paper met the above criteria
   13. glioma ras mapk
   14. 101 search results
   15. 1 paper met the above criteria
   16. ras mutation focal cortical dysplasia
17. 13 search results
18. 2 papers met the above criteria
19. glioma braf mutation
20. 615 search results
21. 1 paper met the above criteria
22. focal cortical dysplasia mutation epilepsy
23. 232 search results
24. 1 paper met the above criteria
   The required information was extracted from the main body of the paper or supplemental documents and analyzed as follows:
1. All the identified variants and their corresponding lesion locations were curated in two separate tables based on their presence in the Ras/Raf/MAPK or PI3K/Akt/mTOR pathways.
2. Lesions outside the cerebral cortex were excluded from further analysis.
3. Lesion locations were reassigned as frontal, parietal, parieto-occipital, occipital, or temporal based on the available information.
   For plotting the cases on the brain in FIG. 1B:
1) The absolute number of cases for each brain region were normalized based on the total number of Ras/Raf/MAPK or PI3K/Akt/mTOR cases.
2) Then the five brain regions were converted to X,Y coordinates and plotted on the surface of a representative drawing of the brain using ggplot2 in RStudio. The original drawing of the brain was obtained from Biorender.com and modified in Adobe Illustrator.
3) The circle diameters for each point represent the normalized case count for each brain region.
   For the bar plot comparing the absolute number of temporal and extra-temporal cases in FIG. 1B:
A) All the frontal, parietal, parieto-occipital, occipital lesions were regrouped as “extra-temporal”.
B) The binomial test was used for statistical comparison of temporal and extra-temporal cases.

### Cell Culture and Plasmid Transfection

KYSE520 (female) cell purchased from Cobioer was cultured in RPMI1640 (Gibco) supplemented with 10% (v/v) FBS (Gibco), 100 units/mL penicillin and 100 mg/mL streptomycin (Gibco). Human HEK293T (female) cell was cultured in DMEM(Gibco) supplemented with 10% (v/v) FBS (Gibco), 100 units/mL penicillin and 100 mg/mL streptomycin (Gibco). All cells were cultured at 37° C. in a humidified atmosphere of 95% air and 5% CO₂. Plasmids were transfected into KYSE520 and HEK 293T cells by using jetOPTIMUS (Polyplus, 117-15) according to the manufacturer's instructions.

### Live Cell Imaging

Cells were grown on 24-well glass bottom plate (Cellvis, P24-1.5H-N) and images were obtained with the Leica TCS SP8 confocal microscopy system using a 100× oil objective (NA=1.4) after transfection. Cells were imaged on a heated stage (37° C.) by heating chamber and supplemented with warmed (37° C.) humidified air. Fluorescent images were processed and assembled into figures using LAS X (Leica) and Fiji.

### Fluorescence Recovery After Photobleaching (FRAP)

KYSE520 Cells were grown on 24-well glass bottom plate (Cellvis, P24-1.5H-N) and the FRAP assay was performed using the FRAP module of the Leica TCS SP8 confocal microscopy system. The Shp2^{mut}-mEGFP was bleached using a 488-nm laser beam. Bleaching was focused on a circular region of interest (ROI) using 100% laser power and time-lapse images were collected. Fluorescence intensity was measured using Fiji. Background intensity was subtracted, and values are reported relative to pre-bleaching time points. The FRAP results were analyzed by GraphPad Prism6.0.

### Protein Expression and Purification

These mutants of Shp2 were generated using standard PCR methods and confirmed by DNA sequencing and were inserted into a pET28a(+) vector with a 6× histidine tag on the N terminus to the constructs. Then, these constructs were transformed into Escherichia coli BL21(DE3) cells and grown in LB medium at 37° C. to an optical density at OD600 of 0.8. The expression of recombinant proteins was induced by 1 mM IPTG at 16° C. for 18h. Cells were harvested by centrifugation at 8000 rpm for 15 min at 4° C. After centrifugation, cells were lysed in buffer containing 25 mM Tris-HCl pH 8.0, 500 mM NaCl and 1 mM PMSF, followed by supercentrifugation at 18000 rpm for 30 min at 4° C. The supernatant was loaded onto a HisTrap FF chelating column (GE healthcare) in 25 mM Tris-HCl pH 8.0, 500 mM NaCl, 1 mM DTT and proteins were eluted with the addition of 300 mM imidazole. Then, fractions containing Shp2 were concentrated and loaded onto a HiLoad 16/600 Superdex 200 pg column (GE Healthcare) using buffer containing 25 mM Tris-HCl pH 8.0, 150 mM NaCl, 2 mM DTT. Fractions were collected according to the results of SDS-PAGE, and then these proteins were concentrated to 10 mg/mL or more, and stored at −80° C.

### In Vitro Liquid-Liquid Phase Separation (LLPS) Assay

For the LLPS assay, the purified Shp2^{wt} or Shp2^{mut} protein at 8 μM was mixed with a LLPS buffer containing 20 mM Tris pH8.0, 10% (w/v) PEG3350 (Sigma) and incubated for 5 min at room temperature. Next, 2 μL of each sample was pipetted onto a glass dish and imaged using a Leica microscope. Under the same conditions, 30 μL of each sample was added into 384-well white polystyrene plate with clear flat bottom, and the value of OD600 was measured by using a Thermo Varioskan™ LUX microplate reader.

### Western Blot

Whole cell lysates were prepared in 3× loading buffer with phosphatase inhibitors (Sigma, P0044 and P5726). Gel electrophoreses of protein lysates were run on 4%-12% NuPAGE Bis-Tris gels (Thermo Fisher, NP0336BOX) at constant voltage of 150V in MES SDS running buffer (Thermo Fisher, NP0002) for 50 min, then transferred to Nitrocellulose membranes using the iBlot gel transfer stack (Thermo Fisher, IB23002), Program 3 for 7 min. Membrane was blocked with 5% (m/v) BSA for 1 hr at room temperature and then incubated with primary antibodies at 4° C. overnight and second antibodies at room temperature for 1 hr. The antibodies were used as follows: pERKI1/2 (CST #4370), ERK1/2 (CST #9102), SH-PTP2 (Santa Cruz #sc7384), and α/β-tubulin (CST #2148).

### Statistical Analysis for Experimental Data

All data were presented as the mean±standard error of mean (SEM) from independent determinations, and statistical analyses were done using the software Graphpad Prism version 6.0 (GraphPad Software, Inc; La Jolla, CA, USA). Differences of means were tested for statistical significance with two/one-tailed Student's t-test.

### Phosphorylated Erk1/2 Staining of MTLE Tissue

Phosphorylated Erk1/2 (pERK1/2) staining on paraffin-embedded archival hippocampal tissue was performed for cases with the following Ras/Raf/MAPK variants: PTPN11 c.1507G>A (p.G503R), PTPN11 c.1508G>T (p.G503V), NF1 c.654+1G>A (altered splicing), KRAS c.35G>A (p.G12D). Additionally, one case without a known Ras/Raf/MAPK pathogenic variant was included as control: EP13101. Immunohistochemistry was performed using the pERK1/2 antibody (CST #4370) at 1:500 dilution.

### Single Cell RNA Sequencing Reanalysis of Human MTLE Hippocampal Tissue

The data from a previously published 10× Genomics 3′ single cell RNA sequencing (scRNA-seq) dataset was downloaded that used surgical hippocampal resections from MTLE patients (Ayhan F, Kulkami A, Berto S, Sivaprakasam K, Douglas C, Lega B C, Konopka G. Resolving cellular and molecular diversity along the hippocampal anterior-to-posterior axis in humans. Neuron [Internet]. Elsevier; 2021 May 28 [cited 2021 June 14]; 0(0). Available from: http://www.cell.com/neuron/abstract/S0896-6273(21)00329-9 PMID: 34051145). The raw sequencing data for subjects A56 and A57 were downloaded since they best represented the age of subjects in the cohort. Then the expression matrices were generated by CellRanger (10× Genomics, version 5.0.0) using the recommended settings. Low quality cells, as determined by <500 expressed genes and >10% mitochondrial DNA were filtered out. Then Seurat V3 (Stuart T, Butler A, Hoffman P, Hafemeister C, Papalexi E, Mauck W M, Hao Y, Stoeckius M, Smibert P, Satija R. Comprehensive Integration of Single-Cell Data. Cell. 2019 Jun. 13; 177(7):1888-1902.e21) was used to perform all the downstream analyses and plotting. Briefly, the expression matrices from the two cases were log-normalized, integrated, dimensionality reduced using the Uniform Manifold Approximation and Projection for Dimension Reduction (UMAP) approach, and clustered. Known gene markers from prior studies (Huang A Y, Li P, Rodin R E, et al. Parallel RNA and DNA analysis after deep sequencing (PRDD-seq) reveals cell type-specific lineage patterns in human brain. Proc Natl Acad Sci USA. 2020 Jun. 23; 117(25):13886-13895) were used to identity all the major cell clusters. Finally, the log-normalized RNA expression levels for PTPN11, KRAS, SOS1, NF1, and BRAF were plotted.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of treating a subject having or having a propensity to develop a seizure disorder, the method comprising administering to the subject an agent that inhibits a component of a Ras/Raf/MapK signaling pathway, wherein the subject is selected by detecting an alteration in the sequence of a polynucleotide encoding a polypeptide that is a component of the Ras/Raf/MapK signalling pathway, wherein the polynucleotide is present in a biological sample of the subject, thereby treating the subject.

2. A method of treating a subject having or having a propensity to develop a seizure disorder associated with a somatic mutation in a Ras/Raf/MapK signaling pathway, the method comprising administering to the subject an agent that inhibits a component of a Ras/Raf/MapK signaling pathway, thereby treating the subject.

3. The method of claim 1, wherein the component of the Ras/Raf/MapK signaling pathway is PTPN11, KRAS, SOS1, BRAF, CBL, LZTR1, PIK3CA, or NF1.

4. A method of treating a subject having or having a propensity to develop a seizure disorder associated with a somatic mutation in a gene selected from the group consisting of PTPN11, FGFR1, KRAS, NF1, BRAF, RASA1, RAF1, RIT1, and CBL, the method comprising administering to the subject an agent that inhibits a component of a Ras/Raf/MapK signaling pathway, thereby treating the subject.

5. The method of claim 1, wherein the seizure disorder is epilepsy, a focal epilepsy subtype, temporal lobe epilepsy, or mesial temporal lobe epilepsy.

6. The method of claim 1, wherein the polynucleotide is genomic DNA, RNA, or cell free DNA.

7. The method of claim 1, wherein the biological sample comprises cerebrospinal fluid, tissue, plasma, or serum.

8. The method of claim 1, wherein the agent is a RAS, RAF, MAPK, BRAF, MEK, RAF or ALK inhibitor.

9. The method of claim 1, wherein the agent is selected from the group consisting of AMG 510, MRTX849, sorafenib, vemurafenib, dabrafenib or encorafenib, PLX8394, LY3009120, belvarafenib, LXH254, rigosertib, trametinib, dabrafenib, encorafenib, binimetinib, trametinib and selumetinib, ulixertinib and ONC201, erlotinib, lapatinib, momelotinib, pan-RAF inhibitor tovorafenib, vemurafenib, dabrafenib, MEK162, RAF265, XL281/BMS-908662, sorafenib, ASP-3026, alectinib (ALECENSA), brigatinib (AP26113), ceritinib (ZYKADIA), CEP-28122, CEP-37440, crizotinib (XALKORI), entrectinib (e.g., NMS-E628, RXDX-101), PF-06463922, TSR-011, X-376, X-396, TNO155, RMC-4630, JAB-3068, RLY-1971, ERAS-601, BBP-398, and combinations thereof.

10. The method of claim 1, wherein the component of the Ras/Raf/MapK signaling pathway is PTPN11/SHP2.

11. The method of claim 10, wherein the alteration in PTPN11/SHP2 is selected from the group consisting of: D61N, D61G, D61Y, A72V, A72S, A72D, A72T, A72P, E76K, E76V, E76G, E76A, Y297S, F285S, F285C, N308D, N308S, A461T, A461G, P491S, P491H, P498 W, P498L, S502L, S502A, G503V, G503A, G503R, M504V, Q506P, T507K, Q510E, Q510P, and Q510H.

12. The method of claim 2, wherein the somatic mutation in a Ras/Raf/MapK signaling pathway is a somatic mutation in PTPN11/SHP2.

13. The method of claim 12, wherein the somatic mutation in PTPN11/SHP2 is selected from the group consisting of: D61N, D61G, D61Y, A72V, A72S, A72D, A72T, A72P, E76K, E76V, E76G, E76A, Y297S, F285S, F285C, N308D, N308S, A461T, A461G, P491S, P491H, P498 W, P498L, S502L, S502A, G503V, G503A, G503R, M504V, Q506P, T507K, Q510E, Q510P, and Q510H.

14. The method of claim 4, wherein the gene is PTPN11.

15. The method of claim 14, wherein the somatic mutation in PTPN11 is selected from the group consisting of: D61N, D61G, D61Y, A72V, A72S, A72D, A72T, A72P, E76K, E76V, E76G, E76A, Y297S, F285S, F285C, N308D, N308S, A461T, A461G, P491S, P491H, P498 W, P498L, S502L, S502A, G503V, G503A, G503R, M504V, Q506P, T507K, Q510E, Q510P, and Q510H.

16. The method of claim 1, wherein the agent is selected from the group consisting of: TNO155, RMC-4630, JAB-3068, RLY-1971, ERAS-601, BBP-398, and combinations thereof.
